SEQUENCE LISTING (54 sequences)

SEQ ID NO: 1 - DNA, 396 bp, Lama pacos
caggctcatgtccagctgcagcagtctggaggaggcttggtgcagcctggggggtctctgagactctcctgtgcagcctctggaagcatcttcagtatctatgccatgggctggtaccgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaacca

SEQ ID NO: 2 - protein, 132 aa, Lama pacos
QAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQP

SEQ ID NO: 3 - DNA, 399 bp, Lama pacos
caggctcatgtccagctgcagcagtctggcggaggcttggtgcagcctggggggtctctgagactctcctgtgcagcctctggattcactttggattattatgccataggctggttccgccaggccccagggaaggagcgtgagggggtctcatgtattagtagtagtggtggtagcacatactatgcagactccgtgaagggccgattcaccatctccagagacaacgccaagaacacggtgtatctgcaaatgaacagcctgaaacctgaggacacagccgtttattactgtgcagtggctcagagctgtctacggggtggggtacgtggaggctactggggccaggggacccaggtcaccgtctcctcagaacccaagacaccaaaaccacaacca

SEQ ID NO: 4 - protein, 133 aa, Lama pacos
QAHVQLQQSGGGLVQPGGSLRLSCAASGFTLDYYAIGWFRQAPGKEREGVSCISSSGGSTYYADSVKGRFTISRDNAKNTVYLQMNSLKPEDTAVYYCAVAQSCLRGGVRGGYWGQGTQVTVSSEPKTPKPQP

SEQ ID NO: 5 - DNA, 378 bp, Lama pacos
caggctgaggtccagctgcaggagtcaggtggaggcttggtgcagcctggggggtctctgagactctcctgtgcagcctctggattcactttggattattatgccataggctggttccgccaggccccagggaaggagcgtgagggggtctcatgtattagcattagtggtggtagcacaaactatgcagactccgtgaagggccgattcaccatctccagagacaacgcccagaacacggtgtatctgcaaatgaacagcctgaaacctgaggacacagccgtttattactgtgcagcaggtcccccgtgcgaccctactcatgggctaattgagtatgactactggggccaggggacccaggtcaccgtctcctca

SEQ ID NO: 6 - protein, 126 aa, Lama pacos
QAEVQLQESGGGLVQPGGSLRLSCAASGFTLDYYAIGWFRQAPGKEREGVSCISISGGSTNYADSVKGRFTISRDNAQNTVYLQMNSLKPEDTAVYYCAAGPPCDPTHGLIEYDYWGQGTQVTVSS

SEQ ID NO: 7 - DNA, 408 bp, Camelus bactrianus
caggctcaggtccagctgcagcagtcaggcggaggcttggtgcagcctggggggtctctgagactctcctgtgcagcctctggattcactttggattattatgccataggctggttccgccaggccccagggaaggagcgtgagggggtcttatgtattagtagtagtggtggtagcacaaactatgcagactccgtgaagggccgattcaccatctccagagacaacgccaagaacacggtgtatctgcaaatgaacagcctgaaacctgaggacacagccgtttattactgtgcagcagatgacctccggtgcggtagtaactggtcgtcttatttccggggttcctggggccaggggacccaggtcaccgtctcctcggaacccaagacaccaaaaccacaacca

SEQ ID NO: 8 - protein, 136 aa, Lama pacos
QAQVQLQQSGGGLVQPGGSLRLSCAASGFTLDYYAIGWFRQAPGKEREGVLCISSSGGSTNYADSVKGRFTISRDNAKNTVYLQMNSLKPEDTAVYYCAADDLRCGSNWSSYFRGSWGQGTQVTVSSEPKTPKPQP

SEQ ID NO: 9 - DNA, 414 bp, Lama pacos
  misc_feature (20)..(20): n is a, c, g, or t
caggctcatgtccagctgcngcagtctggcggaggcttggtgcagcctggggggtctctgagactctcctgtgcagcctctggattcactttggattattatgccataggctggttccgccaggccccagggaaggagcgtgagggggtctcatgtattagtagtagtgatggtagcacatactatgcagactccgtgaagggccgattcaccatctccagagacaacgccaagaacacggtgtatctgcaaatgaacagcctgaaagctgaggacacagccgtttattactgtgcagcagatgacgtcaaatgccgtgcggctactatagcgacccctaggtcctttggtttctggggccaggggacccaggtcaccgtctcctcggaacccaagacaccaaaaccacaacca

SEQ ID NO: 10 - protein, 138 aa, Lama pacos
  misc_feature (7)..(7): Xaa can be any naturally occurring amino acid
QAHVQLXQSGGGLVQPGGSLRLSCAASGFTLDYYAIGWFRQAPGKEREGVSCISSSDGSTYYADSVKGRFTISRDNAKNTVYLQMNSLKAEDTAVYYCAADDVKCRAATIATPRSFGFWGQGTQVTVSSEPKTPKPQP

SEQ ID NO: 11 - DNA, 375 bp, Lama pacos
caggtgcagctcgtggagtccggaggagggtcggtgcagcctggagggtctctgaggctctcatgcgcggccatcgggagcgtcttcaccatgtataccacggcgtggtaccgccagactccaggaaaccttcgcgagttggttgcttctattactgatgagcatagaacaaattacgcggcctccgcggagggccggttcaccatctccagagacaacgccaagcatacggtggatttgcaaatgacgaatttgaaacctgaggacacggccgtgtattactgtaaactcgagcatgatttggggtactatgactactggggtcaggggacccaggtcaccgtctcctcagaacccaagacaccaaaaccacaa

SEQ ID NO: 12 - protein, 125 aa, Lama pacos
QVQLVESGGGSVQPGGSLRLSCAAIGSVFTMYTTAWYRQTPGNLRELVASITDEHRTNYAASAEGRFTISRDNAKHTVDLQMTNLKPEDTAVYYCKLEHDLGYYDYWGQGTQVTVSSEPKTPKPQ

SEQ ID NO: 13 - DNA, 405 bp, Lama pacos
cagttgcagctcgtggagtcggggggaggcatggtgcagcctggggggtctctgagactctcctgtgcagcctctggattcaccttcagtacctatgacatgagctgggtccgccaggctcccgggaaggggcccgagtgggtctcaattattaatgctggtggtggtagcacatactatgcagcctccgtgaagggccgattcgccatctccagagacaacgccaagaacacactgtatctgcaaatgaacaacctgaaacctgaggacacggccctgtattactgtgcgagagtagctagttactactgtcgtggctatgtgtgttcccctccggagtttgactactggggccaggggacccaggtcaccgtctcctcagaacccaagacaccaaaaccacaa

SEQ ID NO: 14 - protein, 135 aa, Lama pacos
QLQLVESGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSSEPKTPKPQ

SEQ ID NO: 15 - DNA, 387 bp, Lama pacos
caggtgcagctcgtggagtctgggggaggcttggtgcaacctggggattctctgacactttcctgtgcagtctctggattcagcttcagtagttataagatgagctgggtccgccagactccaggaaaggggctcgagtgggtctcaagtattaacagtggtggtggtaccacaaactatgcagactccgtgaagggccgattcaccatctccagagacaacgccaagaatatgctgtatctgcaaatgaataaactgaaacctgaggacacggccgtgtattactgtgctcatcgggtagtaactggtcaactagacctttatgattactggggccaggggacccaggtcaccgtctcctcagaacccaagacaccaaaaccacaa

SEQ ID NO: 16 - protein, 129 aa, Lama pacos
QVQLVESGGGLVQPGDSLTLSCAVSGFSFSSYKMSWVRQTPGKGLEWVSSINSGGGTTNYADSVKGRFTISRDNAKNMLYLQMNKLKPEDTAVYYCAHRVVTGQLDLYDYWGQGTQVTVSSEPKTPKPQ

SEQ ID NO: 17 - DNA, 1710 bp, Homo sapiens
atggacccggccgaggcggtgctgcaagagaaggcactcaagtttatgaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 18 - protein, 569 aa, Homo sapiens
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 19 - DNA, 3132 bp, artificial
  YFP/VHH-B8/TrCP fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggacaggctcatgtccagctgcagcagtctggaggaggcttggtgcagcctggggggtctctgagactctcctgtgcagcctctggaagcatcttcagtatctatgccatgggctggtatcgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaaccagcggccgcaggtaccttggcgattatggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 20 - protein, 1043 aa, Artificial
  YFP/VHH-B8/TrCP Fusion Protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKLICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQPAAAGTLAIMDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 21 - DNA, 2196 bp, artificial
  YFP/VHH-B8/TrCP-D3 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggacaggctcatgtccagctgcagcagtctggaggaggcttggtgcagcctggggggtctctgagactctcctgtgcagcctctggaagcatcttcagtatctatgccatgggctggtatcgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaaccagcggccgcaggtaccttggcgattatggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttataa

SEQ ID NO: 22 - protein, 731 aa, Artificial
  YFP/VHH-B8/TrCP-D3 Fusion Protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQPAAAGTLAIMDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSL

SEQ ID NO: 23 - DNA, 1659 bp, artificial
  YFP/VHH-B8/TrCP-D5 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggacaggctcatgtccagctgcagcagtctggaggaggcttggtgcagcctggggggtctctgagactctcctgtgcagcctctggaagcatcttcagtatctatgccatgggctggtatcgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaaccagcggccgcgatgttgcagagagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttataa

SEQ ID NO: 24 - protein, 552 aa, artificial
  YFP/VHH-B8/TrCP-D5 Fusion Protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQPAAAMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSL

SEQ ID NO: 25 - DNA, 1659 bp, artificial
  YFP/TrCP-D5/VHH-B8 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggcctgcagatgttgcagaaagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttaaacctgcaggctcatgtccagctgcagcagtctggaggaggcttggtgcagcctggggggtctctgagactctcctgtgcagcctctggaagcatcttcagtatctatgccatgggctggtatcgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaataa

SEQ ID NO: 26 - protein, 552 aa, artificial
  YFP/TrCP-D5/VHH-B8 fusion protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGLQMLQKDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLNLQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQ

SEQ ID NO: 27 - DNA, 1671 bp, artificial
  YFP/TrCP-D5/VHH-B10 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggcctgcagatgttgcagaaagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttaaacctgcaggtgcagctcgtggagtcggggggaggcatggtgcagcctggggggtctctgagactctcctgtgcagcctctggattcaccttcagtacctatgacatgagctgggtccgccaggctcccgggaaggggcccgagtgggtctcaattattaatgctggtggtggtagcacatactatgcagcctccgtgaagggccgattcgccatctccagagacaacgccaagaacacactgtatctgcaaatgaacaacctgaaacctgaggacacggccctgtattactgtgcgagagtagctagttactactgtcgtggctatgtgtgttcccctccggagtttgactactggggccaggggacccaggtcaccgtctcctcagaacccaagacaccaaaaccacaataa

SEQ ID NO: 28 - protein, 556 aa, artificial
  YFP/TrCP-D5/VHH-B10 fusion protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGLQMLQKDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLNLQVQLVESGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSSEPKTPKPQ

SEQ ID NO: 29 - DNA, 1506 bp, artificial
  YFP/TrCP-D6/VHH-B8 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggcctgcagatgttgcagaaagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctggcggaggtggctctaacctgcaggctcatgtccagctgcagcagtctggaggaggcttggtgcagcctggggggtctctgagactctcctgtgcagcctctggaagcatcttcagtatctatgccatgggctggtatcgccaggctccagggaagcagcgcgagttggtcgcagctattagtagttatggtagcacaaactatgcagactccgtgaagggccgattcaccatctccagagacaacgccaagaacacggtgtatctgcaaatgaacagcctgaaacctgaggacacggccgtctattattgtaatgcagatatagcgactatgaccgcggtaggtgggtttgactactggggccaggggacccaggtcaccgtctcctcagaacccaagacaccaaaaccacaataa

SEQ ID NO: 30 - protein, 501 aa, artificial
  YFP/TrCP-D6/VHH-B8 fusion protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKLICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGLQMLQKDFITALPARGLDHIAENILSYLDAKSLCAAELVCKEWYRVTSDGMLWKKLIERMVRTDSGGGGSNLQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQ

SEQ ID NO: 31 - DNA, 1518 bp, artificial
  YFP/TrCP-D6/VHH-B10 fusion protein coding sequence
atgacgatggacgagcagcagtcgcaggctgtggctccggtctacgtgggcggcatggacgaaaaaaccaccggttggcgtggtggtcacgttgttgaaggtctggctggtgaactggaacagctgcgtgcccgtctggaacaccacccgcagggtcagcgtgaaccgtccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcggacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggcctgcagatgttgcagaaagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctggcggaggtggctctaacctgcaggtgcagctcgtggagtcggggggaggcatggtgcagcctggggggtctctgagactctcctgtgcagcctctggattcaccttcagtacctatgacatgagctgggtccgccaggctcccgggaaggggcccgagtgggtctcaattattaatgctggtggtggtagcacatactatgcagcctccgtgaagggccgattcgccatctccagagacaacgccaagaacacactgtatctgcaaatgaacaacctgaaacctgaggacacggccctgtattactgtgcgagagtagctagttactactgtcgtggctatgtgtgttcccctccggagtttgactactggggccaggggacccaggtcaccgtctcctcagaacccaagacaccaaaaccacaataa

SEQ ID NO: 32 - protein, 505 aa, artificial
  YFP/TrCP-D6/VHH-B10 fusion protein
MTMDEQQSQAVAPVYVGGMDEKTTGWRGGHVVEGLAGELEQLRARLEHHPQGQREPSMVSKGEELFTGVVPILVELDGDVNGHKFSVSGEGEGDATYGKLTLKLICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGLQMLQKDFITALPARGLDHIAENILSYLDAKSLCAAELVCKEWYRVTSDGMLWKKLIERMVRTDSGGGGSNLQVQLVESGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSSEPKTPKPQ

SEQ ID NO: 33 - DNA, 1332 bp, Clostridium botulinum
atgccttttgttaataaacaatttaattataaagatcctgttaatggtgttgatattgcttatattaaaattcctaatgctggtcaaatgcaacctgttaaagcttttaaaattcataataaaatttgggttattcctgaacgtgatacttttactaatcctgaagaaggtgatcttaatcctcctcctgaagctaaacaagttcctgtttcttattatgattctacttatctttctactgataatgaaaaagataattatcttaaaggtgttactaaactttttgaacgtatttattctactgatcttggtcgtatgcttcttacttctattgttcgtggtattcctttttggggtggttctactattgatactgaacttaaagttattgatactaattgtattaatgttattcaacctgatggttcttatcgttctgaagaacttaatcttgttattattggtccttctgctgatattattcaatttgaatgtaaatcttttggtcatgatgttcttaatcttactcgtaatggttatggttctactcaatatattcgtttttctcctgattttacttttggttttgaagaatctcttgaagttgatactaatcctcttcttggtgctggtaaatttgctactgatcctgctgttactcttgctcatgaacttattcatgctgaacatcgtctttatggtattgctattaatcctaatcgtgtttttaaagttaatactaatgcttattatgaaatgtctggtcttgaagtttcttttgaagaacttcgtacttttggtggtcatgatgctaaatttattgattctcttcaagaaaatgaatttcgtctttattattataataaatttaaagatgttgcttctactcttaataaagctaaatctattattggtactactgcttctcttcaatatatgaaaaatgtttttaaagaaaaatatcttctttctgaagatacttctggtaaattttctgttgataaacttaaatttgataaactttataaaatgcttactgaaatttatactgaagataattttgttaatttttttaaagttattaatcgtaaaacttatcttaattttgataaagctgtttttcgtattaatattgttcctgatgaaaattatactattaaagatggttttaatcttaaaggtgctaatctttctactaattttaatggtcaaaatactgaaattaattctcgtaattttactcgtcttaaaaattttactggtctttttgaattttataaacttctttgtgttcgtggtattattccttttaaaactaaatctcttgatgaa

SEQ ID NO: 34 - protein, 444 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDE

SEQ ID NO: 35 - DNA, 1317 bp, Clostridium botulinum
atgccttttgttaataaacaatttaattataaagatcctgttaatggtgttaaaattcctaatgctggtcaaatgcaacctgttaaagcttttaaaattcataataaaatttgggttattcctgaacgtgatacttttactaatcctgaagaaggtgatcttaatcctcctcctgaagctaaacaagttcctgtttcttattatgattctacttatctttctactgataatgaaaaagataattatcttaaaggtgttactaaactttttgaacgtatttattctactgatcttggtcgtatgcttcttacttctattgttcgtggtattcctttttggggtggttctactattgatactgaacttaaagttattgatactaattgtattaatgttattcaacctgatggttcttatcgttctgaagaacttaatcttgttattattggtccttctgctgatattattcaatttgaatgtaaatcttttggtcatgatgttcttaatcttactcgtaatggttatggttctactcaatatattcgtttttctcctgattttacttttggttttgaagaatctcttgaagttgatactaatcctcttcttggtgctggtaaatttgctactgatcctgctgttactcttgctcatgaacttattcatgctgaacatcgtctttatggtattgctattaatcctaatcgtgtttttaaagttaatactaatgcttattatgaaatgtctggtcttgaagtttcttttgaagaacttcgtacttttggtggtcatgatgctaaatttattgattctcttcaagaaaatgaatttcgtctttattattataataaatttaaagatgttgcttctactcttaataaagctaaatctattattggtactactgcttctcttcaatatatgaaaaatgtttttaaagaaaaatatcttctttctgaagatacttctggtaaattttctgttgataaacttaaatttgataaactttataaaatgcttactgaaatttatactgaagataattttgttaatttttttaaagttattaatcgtaaaacttatcttaattttgataaagctgtttttcgtattaatattgttcctgatgaaaattatactattaaagatggttttaatcttaaaggtgctaatctttctactaattttaatggtcaaaatactgaaattaattctcgtaattttactcgtcttaaaaattttactggtctttttgaattttataaacttctttgtgttcgtggtattattccttttaaaactaaatctcttgatgaa

SEQ ID NO: 36 - protein, 439 aa, Clostridium botulinum
MPFVNKQFNYKDPVNGVKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDE

SEQ ID NO: 37 - DNA, 1293 bp, Clostridium botulinum
atgccttttgttaataaacaatttaattataaagatcctgttaatggtgttgatattgcttatattaaaattcctaatgctggtcaaatgcaacctgttaaagcttttaaaattcataataaaatttgggttattcctgaacgtgatacttttactaatcctgaagaaggtgatcttaatcctcctcctgaagctaaacaagttcctgtttcttattatgattctacttatctttctactgataatgaaaaagataattatcttaaaggtgttactaaactttttgaacgtatttattctactgatcttggtcgtatgcttcttacttctattgttcgtggtattcctttttggggtggttctactattgatactgaacttaaagttattgatactaattgtattaatgttattcaacctgatggttcttatcgttctgaagaacttaatcttgttattattggtccttctgctgatattattcaatttgaatgtaaatcttttggtcatgatgttcttaatcttactcgtaatggttatggttctactcaatatattcgtttttctcctgattttacttttggttttgaagaatctcttgaagttgatactaatcctcttcttggtgctggtaaatttgctactgatcctgctgttactcttgctcatgaacttattcatgctgaacatcgtctttatggtattgctattaatcctaatcgtgtttttaaagttaatactaatgcttattatgaaatgtctggtcttgaagtttcttttgaagaacttcgtacttttggtggtcatgatgctaaatttattgattctcttcaagaaaatgaatttcgtctttattattataaatctattattggtactactgcttctcttcaatatatgaaaaatgtttttaaagaaaaatatcttctttctgaagatacttctggtaaattttctgttgataaacttaaatttgataaactttataaaatgcttactgaaatttatactgaagataattttgttaatttttttaaagttattaatcgtaaaacttatcttaattttgataaagctgtttttcgtattaatattgttcctgatgaaaattatactattaaagatggttttaatcttaaaggtgctaatctttctactaattttaatggtcaaaatactgaaattaattctcgtaattttactcgtcttaaaaattttactggtctttttgaattttataaacttctttgtgttcgtggtattattccttttaaaactaaatctcttgatgaa

SEQ ID NO: 38 - protein, 431 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDE

SEQ ID NO: 39 - DNA, 474 bp, Clostridium botulinum
atgccttttgttaataaacaatttaattataaagatcctgttaaatctattattggtactactgcttctcttcaatatatgaaaaatgtttttaaagaaaaatatcttctttctgaagatacttctggtaaattttctgttgataaacttaaatttgataaactttataaaatgcttactgaaatttatactgaagataattttgttaatttttttaaagttattaatcgtaaaacttatcttaattttgataaagctgtttttcgtattaatattgttcctgatgaaaattatactattaaagatggttttaatcttaaaggtgctaatctttctactaattttaatggtcaaaatactgaaattaattctcgtaattttactcgtcttaaaaattttactggtctttttgaattttataaacttctttgtgttcgtggtattattccttttaaaactaaatctcttgatgaa

SEQ ID NO: 40 - protein, 158 aa, Clostridium botulinum
MPFVNKQFNYKDPVKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDE

SEQ ID NO: 41 - DNA, 2547 bp, Clostridium botulinum
aaagctcttaatgatctttgtattaaagttaataattgggatctttttttttctccttctgaagataattttactaatgatcttaataaaggtgaagaaattacttctgatactaatattgaagctgctgaagaaaatatttctcttgatcttattcaacaatattatcttacttttaattttgataatgaacctgaaaatatttctattgaaaatctttcttctgatattattggtcaacttgaacttatgcctaatattgaacgttttcctaatggtaaaaaatatgaacttgataaatatactatgtttcattatcttcgtgctcaagaatttgaacatggtaaatctcgtattgctcttactaattctgttaatgaagctcttcttaatccttctcgtgtttatacttttttttcttctgattatgttaaaaaagttaataaagctactgaagctgctatgtttcttggttgggttgaacaacttgtttatgattttactgatgaaacttctgaagtttctactactgataaaattgctgatattactattattattccttatattggtcctgctcttaatattggtaatatgctttataaagatgattttgttggtgctcttattttttctggtgctgttattcttcttgaatttattcctgaaattgctattcctgttcttggtacttttgctcttgtttcttatattgctaataaagttcttactgttcaaactattgataatgctctttctaaacgtaatgaaaaatgggatgaagtttataaatatattgttactaattggcttgctaaagttaatactcaaattgatcttattcgtaaaaaaatgaaagaagctcttgaaaatcaagctgaagctactaaagctattattaattatcaatataatcaatatactgaagaagaaaaaaataatattaattttaatattgatgatctttcttctaaacttaatgaatctattaataaagctatgattaatattaataaatttcttaatcaatgttctgtttcttatcttatgaattctatgattccttatggtgttaaacgtcttgaagattttgatgcttctcttaaagatgctcttcttaaatatatttatgataatcgtggtactcttattggtcaagttgatcgtcttaaagataaagttaataatactctttctactgatattccttttcaactttctaaatatgttgataatcaacgtcttctttctacttttactgaatatattaaaaatattattaatacttctattcttaatcttcgttatgaatctaatcatcttattgatctttctcgttatgcttctaaaattaatattggttctaaagttaattttgatcctattgataaaaatcaaattcaactttttaatcttgaatcttctaaaattgaagttattcttaaaaatgctattgtttataattctatgtatgaaaatttttctacttctttttggattcgtattcctaaatattttaattctatttctcttaataatgaatatactattattaattgtatggaaaataattctggttggaaagtttctcttaattatggtgaaattatttggactcttcaagatactcaagaaattaaacaacgtgttgtttttaaatattctcaaatgattaatatttctgattatattaatcgttggatttttgttactattactaataatcgtcttaataattctaaaatttatattaatggtcgtcttattgatcaaaaacctatttctaatcttggtaatattcatgcttctaataatattatgtttaaacttgatggttgtcgtgatactcatcgttatatttggattaaatattttaatctttttgataaagaacttaatgaaaaagaaattaaagatctttatgataatcaatctaattctggtattcttaaagatttttggggtgattatcttcaatatgataaaccttattatatgcttaatctttatgatcctaataaatatgttgatgttaataatgttggtattcgtggttatatgtatcttaaaggtcctcgtggttctgttatgactactaatatttatcttaattcttctctttatcgtggtactaaatttattattaaaaaatatgcttctggtaataaagataatattgttcgtaataatgatcgtgtttatattaatgttgttgttaaaaataaagaatatcgtcttgctactaatgcttctcaagctggtgttgaaaaaattctttctgctcttgaaattcctgatgttggtaatctttctcaagttgttgttatgaaatctaaaaatgatcaaggtattactaataaatgtaaaatgaatcttcaagataataatggtaatgatattggttttattggttttcatcaatttaataatattgctaaacttgttgcttctaattggtataatcgtcaaattgaacgttcttctcgtactcttggttgttcttgggaatttattcctgttgatgatggttggggtgaacgtcctctt

SEQ ID NO: 42 - protein, 849 aa, Clostridium botulinum
KALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 43 - protein, 16 aa, Unknown
  Drosophila antennapedia
RQIKIWFQNRRMKWKK

SEQ ID NO: 44 - protein, 11 aa, Human immunodeficiency virus
YGRKKRRQRRR

SEQ ID NO: 45 - protein, 16 aa, artificial
  synthetic construct
RQIKIWFQNRRMKWKK

SEQ ID NO: 46 - protein, 16 aa, artificial
  synthetic contruct
KKWKMRRNQFWIKIQR

SEQ ID NO: 47 - protein, 16 aa, artificial
  synthetic construct
RQIKIWFQNRRMKWKK

SEQ ID NO: 48 - protein, 16 aa, artificial
  synthetic construct
RQIKIWFPNRRMKWKK

SEQ ID NO: 49 - protein, 16 aa, artificial
  synthetic construct
RQPKIWFPNRRMPWKK

SEQ ID NO: 50 - protein, 16 aa, artificial
  synthetic construct
RQIKIWFQNMRRKWKK

SEQ ID NO: 51 - protein, 16 aa, artificial
  synthetic construct
RQIRIWFQNRRMRWRR

SEQ ID NO: 52 - protein, 16 aa, artificial
  synthetic construct
RRWRRWWRRWWRRWRR

SEQ ID NO: 53 - protein, 16 aa, artificial
  synthetic construct
AAVALLPAVLLALLAP

SEQ ID NO: 54 - protein, 27 aa, artificial
  synthetic construct
GALFLGFLGAAGSTMGAWSQPKSKRKV

(19) United States
(12) Patent Application Publication
(10) Pub. No.: US 2010/0278826 A1
(43) Pub. Date: Nov. 4, 2010

(54) Designer Ubiquitin Ligases For Regulation Of Intracellular Pathogenic Proteins

(76) Inventors: Charles B. Shoemaker, North Grafton, MA (US); George A. Oyler, Baltimore, MD (US); Saul Tzipori, Shrewsbury, MA (US)

Correspondence Address: ANTOINETTE G. GIUGLIANO, P.C.;DBA AGG Intellectual Property Law, 100 Cummings Center, Suite 213C, Beverly, MA, 01915, US

(21) Appl. No.: 12/481,889

(22) Filed: Jun. 10, 2009

Related U.S. Application Data
(60) Provisional application No. 61/060,340, filed on Jun. 10, 2008.

(51) Int. Cl. A61K 39/395; C12N 9/00; C07K 16/18; C07H 21/04; C12N 5/071; C12N 15/79; C07K 14/00; A61P 39/00

(52) U.S. Cl. 424/134.1; 435/183; 530/387.2; 536/23.53; 435/325; 435/320.1; 530/324; 530/350; 536/23.1

(57) ABSTRACT

The present invention relates to a designer or recombinant ubiquitin ligase molecule that includes an antibody fragment that is specific for a toxin active fragment, wherein the toxin active fragment is an enzymatically active fragment of one or more toxins or toxin serotypes; and an E3-ligase domain that comprises an E3-ligase or polypeptide that facilitates E2-mediated ubiquitination of the toxin active fragment. In an embodiment, the composition further includes a delivery system that allow the designer ubiquitin ligase to enter the cell. The present invention further includes methods for treating an individual intoxicated with a toxin by administering the designer ubiquitin ligase of the present invention.

### RELATED APPLICATION

This application claims the benefit of U.S. Provisional Application No. 61/060,340, filed Jun. 10, 2008. The entire teachings of the above application are incorporated herein by reference.

### GOVERNMENT SUPPORT

The invention was supported, in whole or in part, by a grant NO1-A1-30050 from National Institute of Allergy and Infectious Diseases. The Government has certain rights in the invention.

### BACKGROUND OF THE INVENTION

Intoxication with bacterial toxins is a serious health and bioterrorism threat problem. Our inability to effectively treat toxin exposure makes certain toxins particularly dangerous agents for terrorist attacks. Treatment options for individuals after toxin infection are limited. For example, once someone is intoxicated with botulinum neurotoxin, the individual is paralyzed for periods up to 4 to 6 months or longer depending on the toxin serotype because the toxin is slow to degrade. During this time the patient is entirely dependent on ventilation assistance.

A need exists for an effective treatment after intoxication. A further need exists for a composition that can inhibit the toxin but also cause the toxin to be degraded to alleviate symptoms of the toxin infection.

### SUMMARY OF THE INVENTION

The present invention relates to a recombinant ubiquitin ligase molecule that includes an antibody fragment that is specific for a toxin active fragment, wherein the toxin active fragment is an enzymatically active fragment of one or more toxins or toxin serotypes; and an E3-ligase domain that comprises an E3-ligase or polypeptide that facilitates E2-mediated ubiquitination of the toxin active fragment. The toxin includes, e.g., botulinum neurotoxin (BoNT) (serotypes A-G), Clostridia difficile toxins A and B (Tcd A and Tcd B), Clostridium Lethal Toxin, Anthrax Lethal Factor (LF), Ricin, Exotoxin A, Diphtheria toxin, Cholera toxin, Tetanus toxin, Shiga toxin, and any combinations thereof. In an embodiment, the recombinant ubiquitin ligase molecule of the present invention includes an antibody fragment (e.g., camelid or shark) that is specific for a light chain of one or more botulinum neurotoxin (BoNT) serotypes. In an embodiment, the antibody fragment is a single chain antibody fragment. In yet another embodiment, the polypeptide that facilitates E2-mediated ubiquitination is an antibody specific to E2. E3 ligase domains include, for example, one or more of the following: RING, HECT, IkB, HIF, U-box, RIBRR, F-box, BTB, TrCP, or DDS2.

In an aspect of the present invention, the isolated polypeptide molecule of the present invention includes a VHH antibody fragment (e.g., a Camelid antibody VHH fragment) or other antibody fragment that is specific for a toxin active fragment, wherein the toxin active fragment is an enzymatically active fragment of one or more botulinum neurotoxin (BoNT) serotypes; a polypeptide translocation domain and cell binding domain binds to the cellular membrane of a cell and delivers the polypeptide molecule into the cell. For example, the translocation cell-binding domain includes atoxic forms of BoNT or heavy chain only of a BoNT serotype, or atoxic forms of Tcd A or Tcd B. Another aspect of the present invention includes an isolated polypeptide molecule having an antibody fragment (e.g., a VHH antibody fragment) that is specific for a toxin active fragment, wherein the toxin active fragment is an enzymatically active fragment of one or more botulinum neurotoxin (BoNT) serotypes; and a BoNT atoxic holotoxin delivery vehicle; wherein the antibody fragment and the BoNT atoxic holotoxin are fused so that the antibody fragment is transported into a target cell by the atoxic BoNT fusion. In another embodiment, the antibody fragment specific for a toxin active domain is fused to an E3-ligase domain which includes an E3-ligase or polypeptide that facilitates E2-mediated degradation of the toxin active fragment.

The present invention further includes another embodiment of the recombinant ubiquitin ligase molecule. In particular, an embodiment of the recombinant ubiquitin ligase consists of the toxin antibody binding fragment and an E3-ligase domain that comprises an E3-ligase or polypeptide that facilitates E2-mediated degradation of the toxin active fragment; and a translocation/cellular binding domain. The molecule includes an antibody fragment that is specific for a toxin active fragment, wherein the toxin active fragment is an enzymatically active fragment of one or more botulinum neurotoxin (BoNT) serotypes; an E3-ligase domain that comprises an E3-ligase or polypeptide that facilitates E2-mediated degradation of the toxin active fragment; and a translocation/cellular binding domain. In an aspect, the translocation/cellular-binding domain binds to target cell and facilitates endocytosis and delivery of the recombinant ubiquitin ligase into the cell. Examples of translocation/cellular binding domain include an antennapedia protein, HIV TAT protein, herpes simplex virus VP22 protein, penetratin-derived peptides, kFGF, human β3 integrin, L- and D-arginine oligomers, SCWKₙ, (LARL)ₙ, HA2; RGD; K₁ 6RGD oligomer; A1kCWK₁₈, DiCWK18, DipaLytic; Plae, Kplae, MPG peptide, Pep-1, or an atoxic neurotoxin.

The present invention encompasses a Camelid VHH antibody that is specific to the BoNT/A or BoNT/B light chain. In another embodiment, the present invention relates to an isolated antibody that is specific to the BoNT/A light chain, wherein the antibody has any one of the following sequences: an amino acid sequence encoded by a nucleic acid molecule having a sequence of SEQ ID NO: 1, 3, 5, 7, 9, or 11; an amino acid sequence encoded by a complement of SEQ ID NO: 1, 3, 5, 7, 9, or 11; an amino acid sequence encoded by a nucleic acid molecule that hybridizes to SEQ ID NO: 1, 3, 5, 7, 9, or 11 under high stringency conditions; and an amino acid sequence of SEQ ID NO: 2, 4, 6, 8, 10, or 12. The present invention further relates to an isolated nucleic acid molecule that encodes an isolated antibody that is specific to the BoNT/A light chain, wherein the antibody is encoded by one of the following nucleic acid molecule having a sequence: SEQ ID NO: 1, 3, 5, 7, 9, or 11; a complement of SEQ ID NO: 1, 3, 5, 7, 9, or 11; that hybridizes to SEQ ID NO: 1, 3, 5, 7, 9, or 11 under high stringency conditions; and that encodes SEQ ID NO: 2, 4, 6, 8, 10, or 12. The present invention further pertains to sequences having greater than or equal to about 70% identity or similarity with said sequences. The present invention further includes host cell, vectors, plasmids and viruses having the sequences of the present invention.

An embodiment of the present invention includes an isolated polypeptide, wherein the polypeptide includes a Camelid VHH antibody domain that is specific to the BoNT/A light chain, wherein the Camelid VHH antibody domain has a sequence that comprises one of the following an amino acid sequences: an amino acid sequence encoded by a nucleic acid molecule having a sequence of SEQ ID NO: 1, 3, 5, 7, 9, or 11; an amino acid sequence encoded by a complement of SEQ ID NO: 1, 3, 5, 7, 9, or 11; an amino acid sequence encoded by a nucleic acid molecule that hybridizes to SEQ ID NO: 1, 3, 5, 7, 9, or 11 under high stringency conditions; and an amino acid sequence of SEQ ID NO: 2, 4, 6, 8, 10, or 12; and a BoNT domain that includes one of the following an amino acid sequences: an amino acid sequence encoded by a nucleic acid molecule having a sequence of SEQ ID NO: 33, 35, 37, or 39; an amino acid sequence encoded by a complement of SEQ ID NO: 33, 35, 37, or 39; an amino acid sequence encoded by a nucleic acid molecule that hybridizes to SEQ ID NO: 33, 35, 37, or 39; and an amino acid sequence of SEQ ID NO: 34, 36, 38, or 40.

In another embodiment, the present invention relates to an isolated antibody that is specific to the BoNT/B light chain, wherein the antibody has any one of the following sequences: an amino acid sequence encoded by a nucleic acid molecule having a sequence of SEQ ID NO: 13 or 15; an amino acid sequence encoded by a complement of SEQ ID NO: 13 or 15; an amino acid sequence encoded by a nucleic acid molecule that hybridizes to SEQ ID NO: 13 or 15; under high stringency conditions; and an amino acid sequence of SEQ ID NO: 14 or 16. The present invention further relates to an isolated nucleic acid molecule that encodes an isolated antibody that is specific to the BoNT/A light chain, wherein the antibody is encoded by one of the following nucleic acid molecule having a sequence: SEQ ID NO: 13 or 15; a complement of SEQ ID NO: 13 or 15; that hybridizes to SEQ ID NO: 13 or 15 under high stringency conditions; and that encodes SEQ ID NO: 14 or 16. The present invention further pertains to sequences having greater than or equal to about 70% identity or similarity with said sequences. The present invention further includes host cell, vectors, plasmids and viruses having the sequences of the present invention.

An embodiment of the present invention includes an isolated polypeptide, wherein the polypeptide includes a Camelid VHH antibody domain that is specific to the BoNT/B light chain, wherein the Camelid VHH antibody domain has a sequence that comprises one of the following an amino acid sequences: an amino acid sequence encoded by a nucleic acid molecule having a sequence of SEQ ID NO: 13 or 15; an amino acid sequence encoded by a complement of SEQ ID NO: 13 or 15; an amino acid sequence encoded by a nucleic acid molecule that hybridizes to SEQ ID NO: 13 or 15 under high stringency conditions; and an amino acid sequence of SEQ ID NO: 14 or 16; and a BoNT domain that includes one of the following an amino acid sequences: an amino acid sequence encoded by a nucleic acid molecule having a sequence of SEQ ID NO: 33, 35, 37, or 39; an amino acid sequence encoded by a complement of SEQ ID NO: 33, 35, 37, or 39; an amino acid sequence encoded by a nucleic acid molecule that hybridizes to SEQ ID NO: 33, 35, 37, or 39; and an amino acid sequence of SEQ ID NO: 34, 36, 38, or 40.

The present invention further relates to an isolated polypeptide, wherein the polypeptide has a sequence that comprises one or more of the following an amino acid sequences: an amino acid sequence encoded by a nucleic acid having greater than or equal to about 70% identity with SEQ ID NO: 19, 21, 23, 25, 27, 29, 31, 35, 37, 39, 41; an amino acid sequence encoded by a complement having greater than or equal to about 70% identity with of SEQ ID NO: 19, 21, 23, 25, 27, 29, 31, 35, 37, 39, 41; an amino acid sequence encoded by a nucleic acid molecule having greater than or equal to about 70% identity with a molecule that hybridizes to SEQ ID NO: 19, 21, 23, 25, 27, 29, 31, 35, 37, 39, 41; or an amino acid sequence having greater than or equal to about 70% similarity to a sequence set forth in SEQ ID NO: 20, 22, 24, 26, 28, 30, 32, 36, 38, 40, 42. Similarly, the present invention pertains to an isolated nucleic acid molecule, wherein the nucleic acid molecule has a sequence that comprises one or more of the following nucleic acid sequences: a nucleic acid sequence having greater than or equal to about 70% identity with SEQ ID NO: 19, 21, 23, 25, 27, 29, 31, 35, 37, 39, 41; a nucleic acid sequence complement having greater than or equal to about 70% identity with of SEQ ID NO: 19, 21, 23, 25, 27, 29, 31, 35, 37, 39, 41; a nucleic acid molecule having greater than or equal to about 70% identity with a molecule that hybridizes to SEQ ID NO: 19, 21, 23, 25, 27, 29, 31, 35, 37, 39, 41; or a nucleic acid sequence that encodes an amino acid sequence having greater than or equal to about 70% similarity to a sequence set forth in SEQ ID NO: 20, 22, 24, 26, 28, 30, 32, 36, 38, 40, 42.

The present invention further pertains to methods of degrading or inhibiting one or more of the BoNT serotypes active light chain domain that have intoxicated one or more cells. The steps of the method include contacting an amount of the recombinant ubiquitin ligase of the present invention with the intoxicated cells; wherein the recombinant ubiquitin ligase degrades or inhibits at least one BoNT serotype. The amount of recombinant ubiquitin ligase that comes into contact with the intoxicated cell ranges from about 1 pM to about 100 mM. The time of said contact ranges from about 30 minutes to about 1 week.

The present invention also relates to methods of treating an individual having one or more cells intoxicated with one or more BoNT serotypes. The methods include administering to the individual an amount of recombinant ubiquitin ligase of the present invention in a carrier; wherein one or more symptoms associated with BoNT intoxication are reduced or reversed. In an aspect, one or more of the following symptoms associated with BoNT intoxication are reduced: blurred vision, dry mouth, difficulty swallowing, difficulty speaking, paralysis, muscle weakness, and respiratory failure. The recombinant ubiquitin ligase of the present invention can be administered intravenously, parenterally, orally, nasally, by inhalation, by implant, by injection, or by suppository. The amount of recombinant ubiquitin ligase can be administered once or periodically. Yet another aspect of the present invention relates to a pharmaceutical composition having the recombinant ubiquitin ligase molecule of the present invention, and a carrier. In yet another embodiment of the invention, one or more cells intoxicated with one or more BoNT serotypes is treated by expression of the recombinant ubiquitin ligase molecule from a nucleic acid vector encoding the protein sequence of the recombinant ubiquitin ligase such as via a viral vector.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic showing an embodiment of the ubiquitin designer ligase framework with and without a delivery vehicle.

FIG. 2 is a schematic showing photographs of Western Blots probed with an anti-XFP antibody recognizing the YFP component or an Anti-SNAP25antibody. The results are from an experiment in which Neuro2a cells were transfected with vector control or expression plasmids designed for expression of YFP/VHH-B8 alone or the designer ligases, YFP/VHH-B8/RING, YFP/VHH-B8/TrCP. Cells were co-transfected with the following expression plasmids for the CFP/A-LC protein: Lane 1: CFP-A/Lc; Lane 2: CFP-A/Lc+YFP-B8; Lane 3: CFP-A/Lc+YFP-B8-RING; Lane 4: CFP-A/Lc+YFP-B8-TrCP; Lane 5: YFP-B8-TrCP; M: Marker.

FIG. 3 is a graph of Camelid VHH B8's inhibition of the BoNT/A LC as a function of SNAP25 cleavage.

FIG. 4 shows photographs of a Camelid VHH antibody co-localized with BoNT/A LC.

FIG. 5 is a schematic showing a photograph of a Western Blot probed with an anti-XFP antibody. The figure shows results from an experiment in which Neuro2a cells were transfected with expression plasmids encoding a vector control (lanes 1 and 2) or the designer ligase YFP/VHH-B8/TrCP (lanes 3 and 4). At the same time, cells were co-transfected with an expression plasmid for the YFP/SNAP25/CFP fusion protein.

FIG. 6 is schematic showing a representation of the series of deletions and inversions that were generated and tested for efficacy as designer ligases. B8 is the anti-BoNT/A Lc VHH-B8 and B10 is the anti-BoNT/B Lc VHH-B10.

FIG. 7 a photograph of a Western Blot probed with an anti-XFP antibody. The figure shows results from an experiment in which Neuro2a cells were transfected with expression plasmids encoding the following designer ligases: B8/D3, B8/D4, B8/-D5, D3/B8, and D5/B8. A day later, extracts were prepared and incubated with GST/A-LC and bound protein purified by glutathione affinity.

FIG. 8 is a schematic showing photographs of Western Blots probed with anti-A-LC, anti-B-LC mAbs or anti-SNAP25 polyclonal antibodies. The figure shows results from an experiment in which neuroblastoma cells were co-transfected with expression plasmids encoding the designer ligases (D5/B8, D5/B10, D6/B8, D6/B10) and either CFP/A-LC or CFP/B-LC.

FIG. 9 is a schematic showing a designer E3 designer ligase for therapeutic development.

FIGS. 10A-10B show the sequences of eight Camelid VHH antibodies that are specific to the light chain of Botulinum Neurotoxin serotypes: Camelid VHH B8 nucleic acid (SEQ ID NO:1) and amino acid sequence (SEQ ID NO:2), Camelid VHH G6 nucleic acid (SEQ ID NO:3) and amino acid sequence (SEQ ID NO:4), Camelid VHH A6 nucleic acid (SEQ ID NO:5) and amino acid sequence (SEQ ID NO: 6), Camelid VHH D4 nucleic acid (SEQ ID NO:7) and amino acid sequence (SEQ ID NO: 8), Camelid VHH E3 nucleic acid (SEQ ID NO:8) and amino acid sequence (SEQ ID NO:10), Camelid VHH H7 nucleic acid (SEQ ID NO:11) and amino acid sequence (SEQ ID NO:12), Camelid VHH B10 nucleic acid (SEQ ID NO:13) and amino acid sequence (SEQ ID NO:14), Camelid VHH C3 nucleic acid (SEQ ID NO:15) and amino acid sequence (SEQ ID NO:16).

FIGS. 11A-11F show the full length an E3 ligase domain, TrCP, nucleic acid sequence (SEQ ID NO: 17) and amino acid sequence (SEQ ID NO: 18). The figure also includes the sequences of following fusion proteins: YFP/VHH-B8/TrCP nucleic acid (SEQ ID NO: 19) and amino acid (SEQ ID NO: 20), YFP/VHH-B8/TrCP-D3 nucleic acid (SEQ ID NO: 21) and amino acid (SEQ ID NO: 22), YFP/VHH-B8/TrCP-D5 nucleic acid (SEQ ID NO: 23) and amino acid (SEQ ID NO: 24), YFP/TrCP-D5/VHH-B8 nucleic acid (SEQ ID NO: 25) and amino acid (SEQ ID NO: 26), YFP/TrCP-D5/VHH-B10 nucleic acid (SEQ ID NO: 27) and amino acid (SEQ ID NO: 28), YFP/TrCP-D6/VHH-B8 nucleic acid (SEQ ID NO: 29) and amino acid (SEQ ID NO: 30), YFP/TrCP-D6/VHH-B10 nucleic acid (SEQ ID NO: 31) and amino acid (SEQ ID NO: 32).

FIGS. 12A-D show the nucleic acid (SEQ ID NO: 33) sequence and the amino acid sequence (SEQ ID NO: 34) for the light chain of the BoNT/A sequence, the nucleic acid (SEQ ID NO: 35) sequence and the amino acid sequence (SEQ ID NO: 36) for deletion 1 thereof; the nucleic acid (SEQ ID NO: 37) sequence and the amino acid sequence (SEQ ID NO: 38) for deletion 2 thereof; the nucleic acid (SEQ ID NO: 39) sequence and the amino acid sequence (SEQ ID NO: 40) for deletion 3 thereof the nucleic acid (SEQ ID NO: 41) sequence and the amino acid sequence (SEQ ID NO: 42 for the heavy chain of the BoNT/A sequence and deletion thereof.

### DETAILED DESCRIPTION OF THE INVENTION

A description of preferred embodiments of the invention follows.

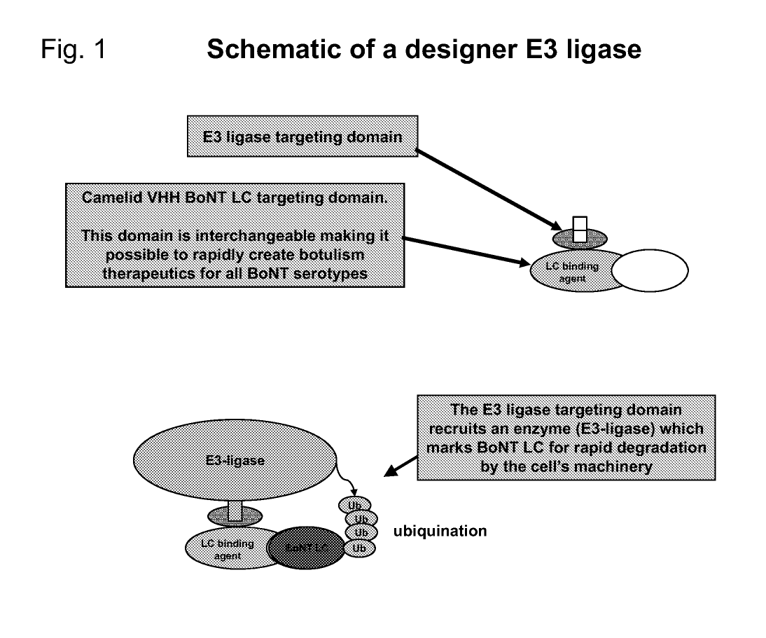

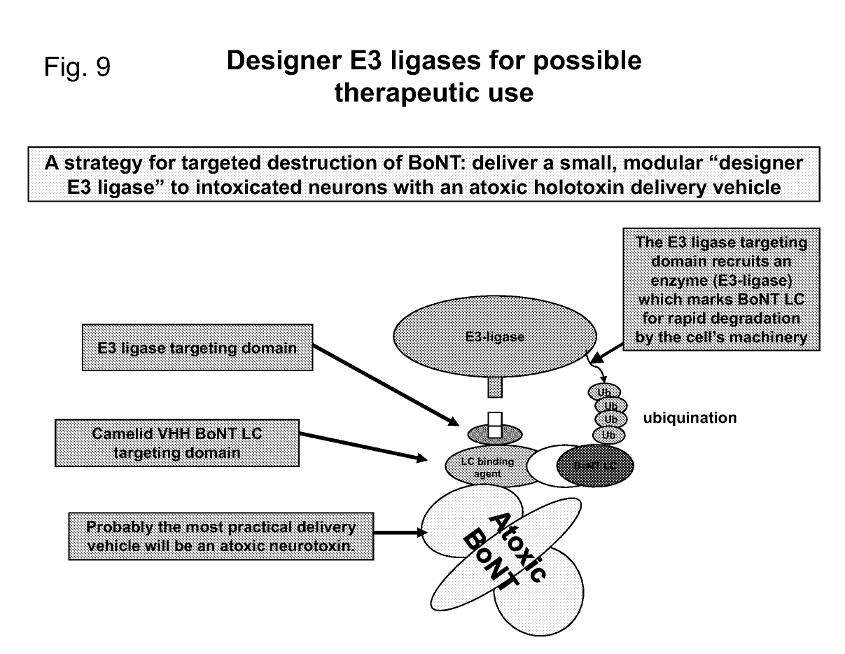

The present invention relates to a recombinant ubiquitin ligase molecule, also referred to herein as a “designer ubiquitin ligase”. In one aspect, the molecule of the present invention has at least two domains: an antibody fragment domain that is specific for the enzymatically active portion of a toxin, referred to herein as the “toxin active fragment”, and an E3 ligase domain that facilitates E2-mediated ubiquitination (FIG. 1). Such a designer ligase, in one aspect, allows the molecule to bind to the enzymatically active portion of the toxin, inhibiting its action, while the E3 ligase domain promotes the ubiquitination and subsequent degradation of the toxin. In another embodiment, the composition of the present invention includes another domain, a cargo carrying component, usually an atoxic fragment of a toxin's enzymatically active domain, a translocation component, and a cell binding component, which allows the molecule to bind to the cellular membrane and then be transported into the cell e.g., a delivery vehicle (see FIG. 9). Accordingly, the present invention relates to the composition as well as methods for inhibiting and/or degrading one or more toxins.

### The Antibody Fragment Specific for the Enzymatically Active Portion of the Toxin:

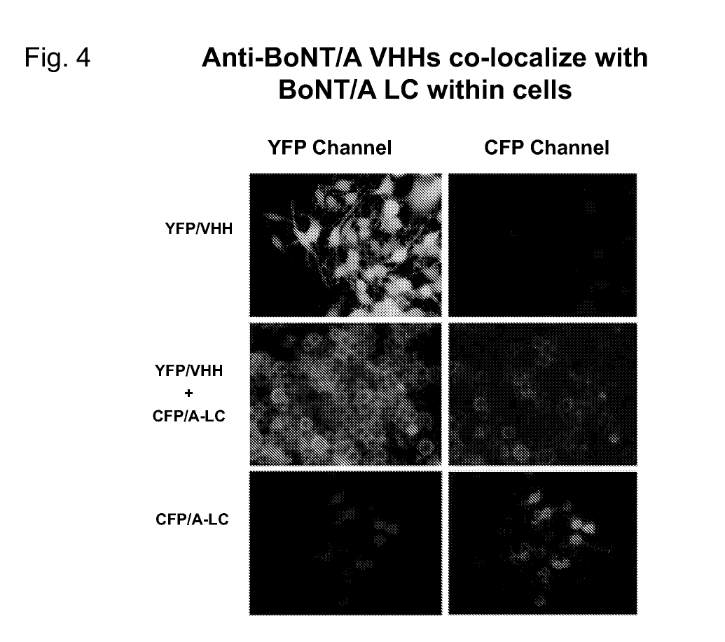

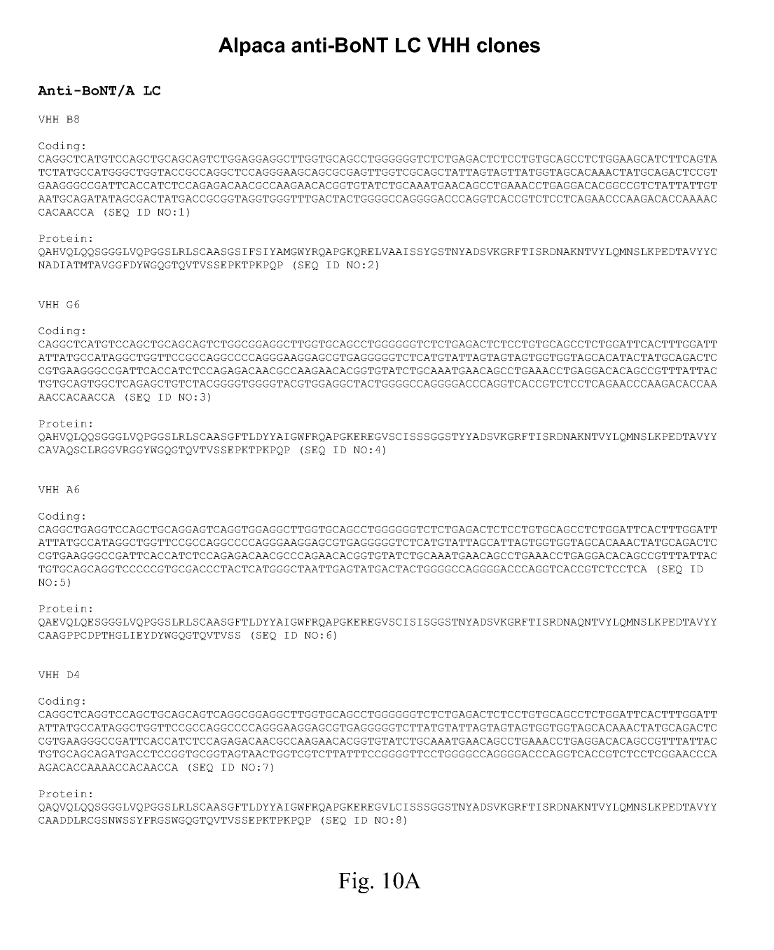

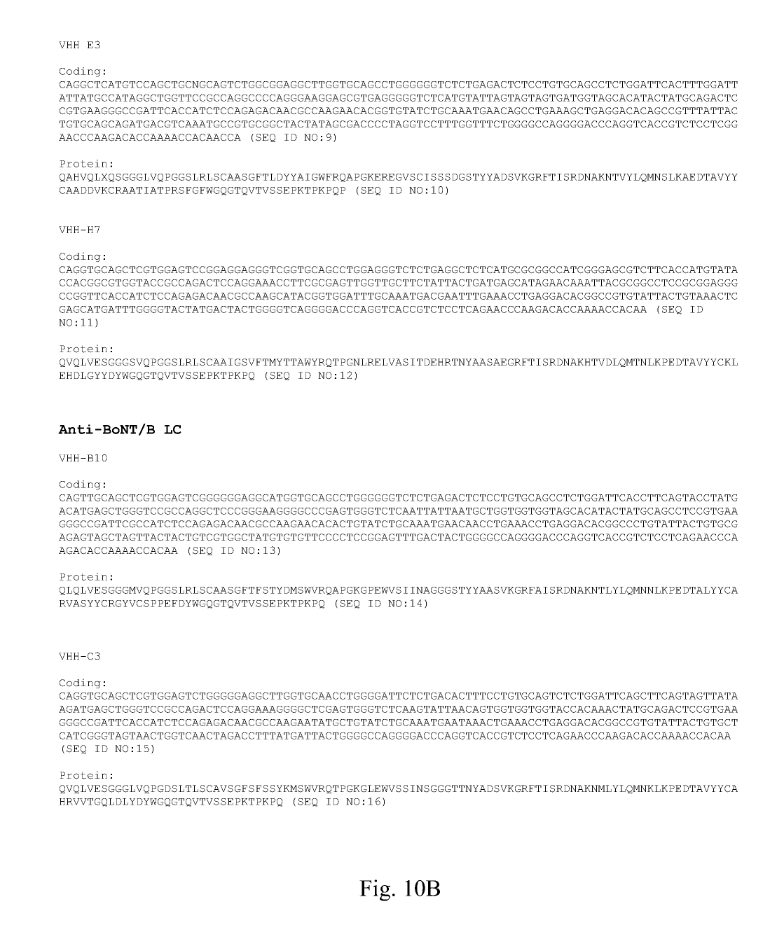

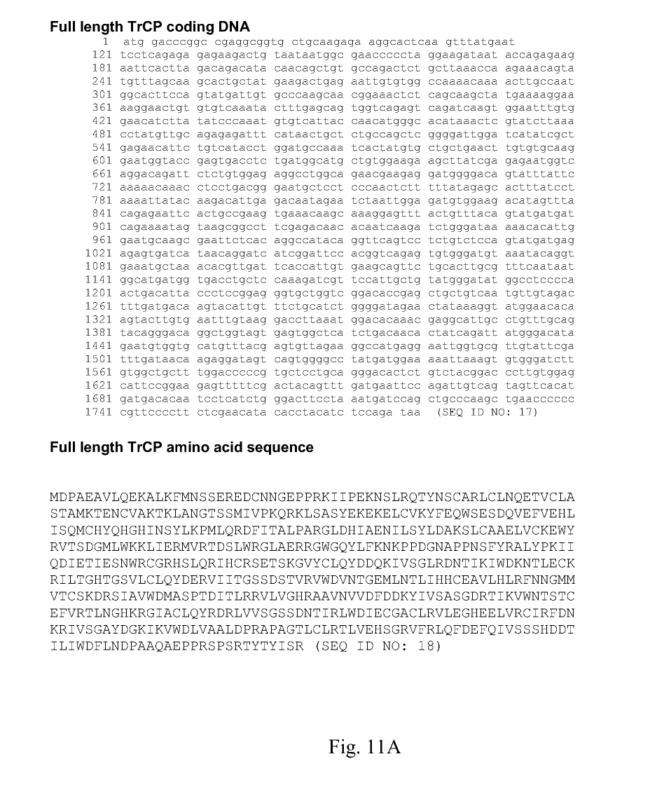

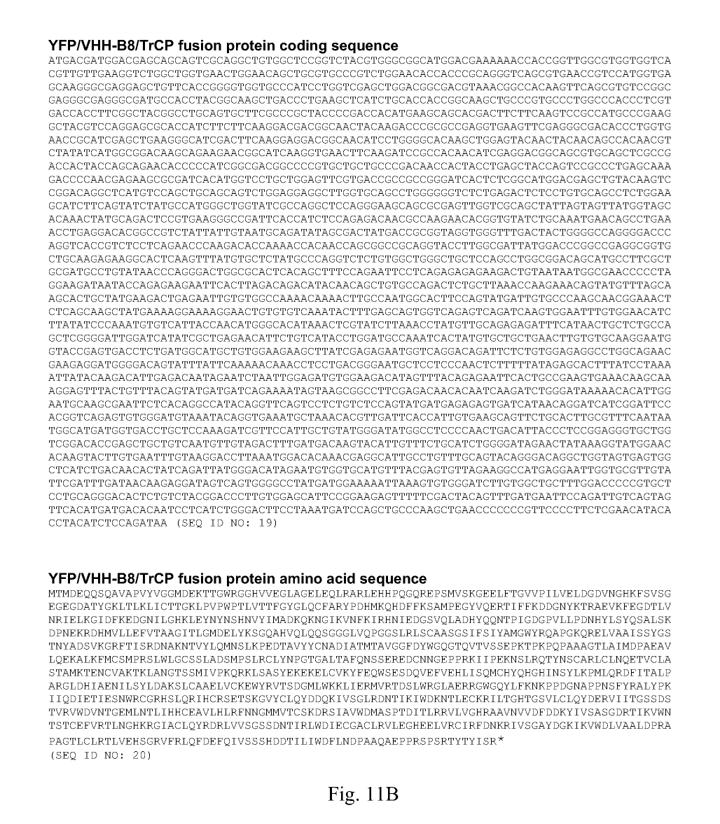

The present invention relates to an antibody fragment that is specific to a portion of the toxin. The term “antibody fragment” refers to a portion of an immunoglobulin having specificity and affinity to the enzymatically active portion of a toxin or a molecule involved with its function. The term, “antibody fragment”, is intended to encompass fragments from both polyclonal and monoclonal antibodies including transgenically produced antibodies, single-chain antibodies (scFvs), recombinant Fabs, heavy-chain-only antibodies, and specifically recombinant shark or camelid single chain antibodies (VHHs). Camelid VHHs are also referred to as nanobodies and several were made to the light chain of BoNT/A and BoNT/B and are referred to as B8, G6, A6, D4, E3, and H7, B10 and C3 respectively (see FIGS. 10A-B, SEQ ID NO: 1-16. The Camelid VHH antibody specific for the light chain of BoNT/A with the highest affinity was B8 (see FIG. 3). The camelid VHHs are functionally capable of binding to BoNT LCs even within eukaryotic cells (FIG. 4).

Nanobodies are antibody-derived therapeutic proteins that contain the unique structural and functional properties of naturally-occurring heavy-chain antibodies. The Nanobody technology is based on fully functional antibodies from Camelids that lack light chains. These heavy-chain only antibodies contain a single variable domain (VHH) and two constant domains (CH2 and CH3). The cloned and isolated Camelid VHH domain is a stable polypeptide harboring the antigen-binding capacity of the original heavy-chain antibody.

Suitable methods of producing or isolating antibody fragments of the requisite specificity are known in the art and include for example, methods which select recombinant antibody from a library created by PCR of the DNA encoding the antigen binding regions.

Functional fragments of antibodies, including fragments of chimeric, humanized, primatized, veneered or single chain antibodies, can also be produced. Functional fragments or portions of the foregoing antibodies include those which are reactive with the enzymatically active portion of the toxin. For example, antibody fragments capable of binding to the enzymatically active portion of the toxin, including, but not limited to scFvs, Fabs, Camelid VHHs, Fv, Fab, Fab′ and F(ab′)₂ are encompassed by the invention. Such fragments can be produced by enzymatic cleavage or by recombinant techniques. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain. Accordingly, the present invention encompasses a polynucleic acid that encodes the antibody domain described herein.

The antibody-binding domain includes any antagonist that binds to the enzymatically active portion of the toxin, or inhibits function thereof. Known antagonists, or those developed in the future, can be used with the present invention.

### The Toxin:

The present invention relates to an antibody or antagonist that inhibits or binds to the enzymatically active portion of a toxin. The enzymatically active portion of the toxin is referred to herein as a “toxin active fragment” or in the case of BoNT it is referred to as the light chain. The toxin active fragment can be from any toxin known or later discovered or developed. A toxin is often a molecule, generally produced by a living cell or organism, and can enter a cell and causes disease or injury. Certain toxins come from animals such as spiders, snakes, pufferfish, scorpions, jellyfish, and bees while many others come from fungi, bacterial and plants. These types of toxins can affect various tissues including the nervous system e.g. neurotoxins (e.g., Botulinum neurotoxin). A common effect is paralysis such as that caused by botulinum neurotoxin (BoNT). In addition to the symptoms of intoxication, in certain embodiments, the duration of action of the toxin may be an important factor to consider in developing treatments. The duration of action of BoNT can be quite long. Shortening this duration of intoxication is the goal of the treatment described in this invention. Other protein toxins implicated by the invention are Tcd A and Tcd B, Clostridium Lethal Toxin, Anthrax Lethal Factor, Ricin, Exotoxin A, Diphtheria toxin, Cholera toxin, Tetanus toxin, Shiga toxin, and a combination thereof.

In an embodiment, BoNT or an enzymatically active portion thereof is the target of the treatment. BoNT is a neurotoxin protein produced by the bacterium, Clostridium botulinum. There are at least seven different BoNT serotypes (A to G), and some of the serotypes have various isotypes (e.g., three isotypes of serotype A have been described). Generally, the BoNT has two chains, an enzymatically active light chain (e.g., about 50-kDa) and a heavy chain (e.g., about 100-kDa) often joined by a peptide or disulfide bond. The heavy chain is formed by a translocation and a cell-binding domain that allows for the toxin to bind to and enter the cell and then translocate the active light chain fragment into the cytosol of the cell. The light chain is a proteolytic enzyme that cleaves a vesicle fusion protein (e.g., SNAP-25, syntaxin or synaptobrevin) in the motor neuron presynaptic terminal at a neuromuscular junction, preventing vesicles from releasing acetylcholine. By inhibiting acetylcholine release, the toxin interferes with nerve impulses ability to cause muscle contraction and results in the paralysis of muscles seen in botulism.

The antibody domain can be specific to a toxin active fragment that has been derivative from a toxin's enzymatically active portion. A “derivative” refers to a molecule with toxin enzymatic activity but contains one or more chemical or functional alterations thereof, as compared to the native enzymatic portion. For instance, the botulinum toxin light chain can be modified so that one or more of its amino acid residues is deleted, modified, replaced, or truncated. For instance, the botulinum toxin light chain can be modified in a way such that the modification enhances its properties or decreases undesirable side effects, but that still retains the desired botulinum toxin activity. The botulinum toxin can be derived from any of the botulinum toxin serotypes and/or isoforms produced by the bacterium. Alternatively, the botulinum toxin can be a toxin prepared using recombinant or synthetic chemical techniques (e.g., a recombinant peptide, a fusion protein, or a hybrid neurotoxin, as prepared from subunits or domains of different botulinum toxin serotypes). Additionally, the botulinum toxin active fragment can be in the form of a botulinum toxin precursor, which can itself be non-toxic, for instance a non-toxic zinc protease that becomes toxic on proteolytic cleavage.

“Enzymatically active” portion of the toxin refers to the portion of the toxin that normally gets inside of the cell (e.g., in the endosome or cytosol) and has enzymatic activity. Toxins are often made up of at least two parts, a cell-binding/translocation domain, and an enzymatically active domain. In the BoNT, the enzymatically active domain is often referred to as the “light chain.” However, the enzymatically active domain for other toxins can have other names. For example, with the ricin toxin, the enzymatically active domain is the “A” Chain. The cell-binding/translocation domain facilitates binding of the toxin active fragment to the cell membrane and transporting the fragment across the cellular membrane. For certain toxins like the BoNT, this domain is referred to as the heavy chain. For other toxins, such as ricin, this is referred to as the B Chain.

In an embodiment, enzymatically active refers to a protein that causes the cleavage of one or more proteins in the cell, which in turn causes toxic effects. In the case of certain toxins, the enzymatically active domain cleaves a SNARE (“Soluble NSF Attachment Receptor”) protein. SNARE proteins are a large protein superfamily consisting of several members. The primary role of SNARE proteins is to mediate fusion of cellular transport vesicles with the cell membrane. The core SNARE complex is formed by four α-helices contributed by synaptobrevin, syntaxin and SNAP-25. Different toxins, serotypes of a certain toxin, or cell types will involve cleavage of different SNARE proteins. Tetanospasmin, e.g., is the neurotoxin produced by Clostridium tetani and causes tetanus. BoNT A, C, and E cleave SNAP-25, in addition BoNT C cleaves syntaxin 1. BoNT B, D, F, G and tetanus toxin cleave VAMP-2. More than one SNARE protein can be cleaved by a single toxin active fragment.

Botulinum toxin is a zinc-dependent protease and enzymatic activity resides generally in the light chain of the molecules. These enzymes cleave SNARE proteins, synaptobrevin 1 and 2, syntaxin and SNAP 25, which form the core of a complex involved in the fusion of transmitter-containing vesicles with the plasma membrane. Prior to fusion, the SNARE proteins in the vesicle and plasma membrane interact forming a complex which contracts with an increase in the intracellular calcium concentration, pulling the vesicle close to the plasma membrane. Interaction between lipids in the two membranes allows the vesicle and nerve terminal active zone to fuse. During this fusion, the contents of the vesicles, mainly neurotransmitters, are released, and the inner surface of the vesicles is exposed to the synaptic cleft. If one of the SNARE proteins is cleaved by a neurotoxin, complex formation cannot occur and fusion is interrupted.

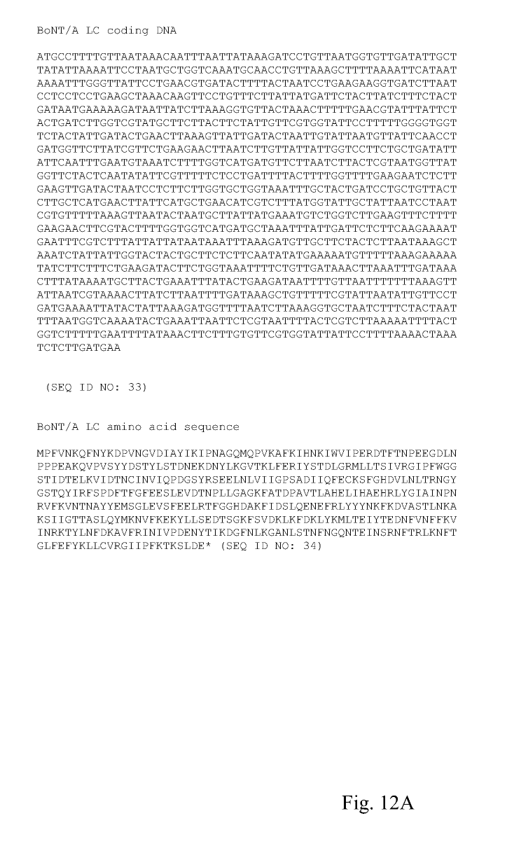

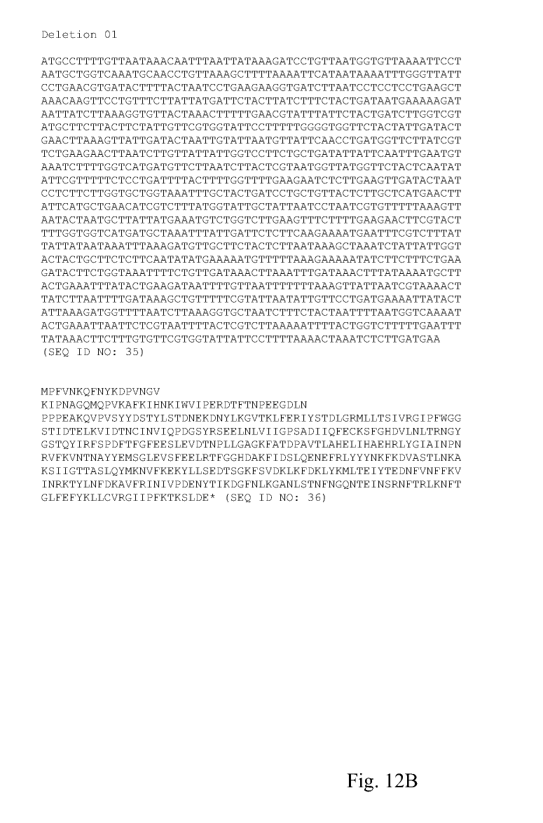

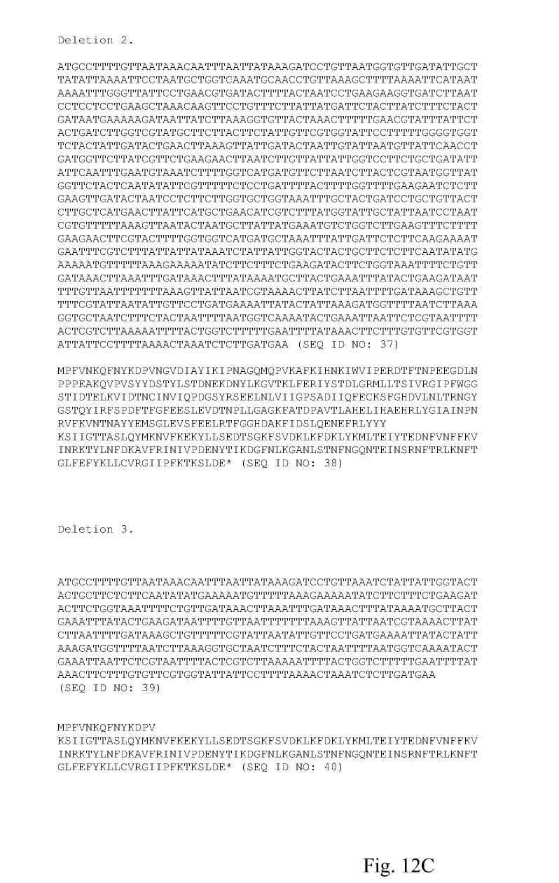

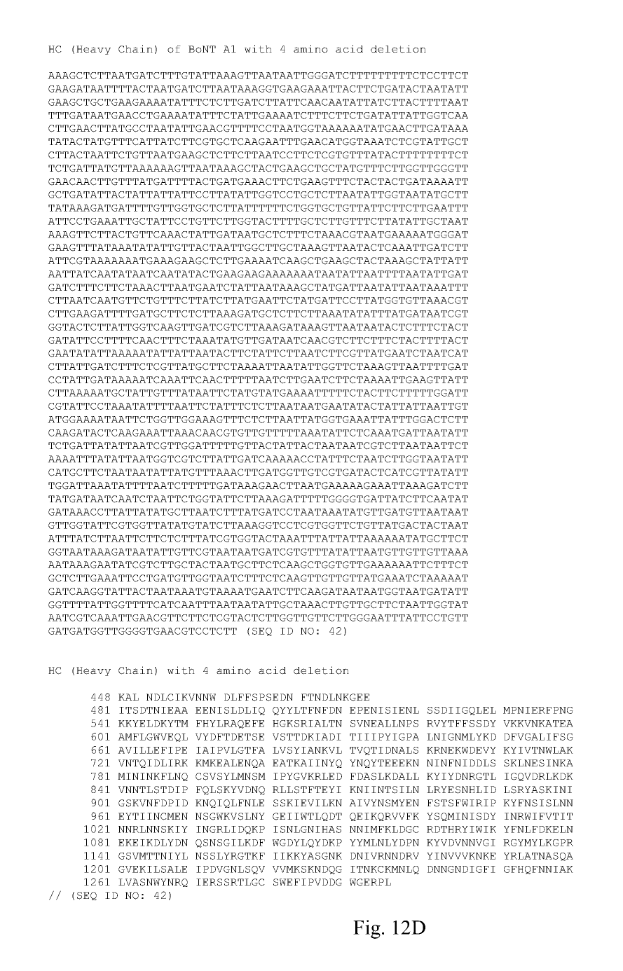

With respect to the BoNT serotypes, the light chain for serotype A has an amino acid sequence, or is encoded by a nucleic acid sequence as shown in FIGS. 12A-12D. The present invention specifically relates to an antibody that is specific to the light chain of any of the BoNT serotypes, as well as any recombinant, mutated, truncated or deleted portions thereof. Several Camelid VHH antibodies to BoNT/A and BoNT/B serotypes were identified, and their sequences are shown in FIGS. 10A-B. As such, the toxin active fragment can be the recombinant form of any enzymatically active portion thereof (e.g., the light chain of a BoNT serotype).

The antibody fragment can be made from recombinant DNA which transcribes the desired amino acid sequence that is specific to the toxin active fragment. The recombinant nucleic acid sequence can be a nucleotide “variant” of an antibody to any enzymatically active portion of a toxin. A variant is a sequence that differs from the known nucleotide sequence for that molecule in having a truncation, and/or one or more nucleotide deletions, substitutions or additions. Such modifications can be readily introduced using standard mutagenesis techniques, such as oligonucleotide-directed site-specific mutagenesis as taught, for example, by Adelman et al. (DNA 2:183, 1983). Nucleotide variants can be naturally occurring allelic variants, or non-naturally occurring variants. Variant nucleotide sequences preferably exhibit at least about 70%, more preferably at least about 80% and most preferably at least about 90% homology to the recited sequence. Such variant nucleotide sequences will generally hybridize to the recited nucleotide sequence under stringent conditions. In one embodiment, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C. The specific sequences, variants of the antibody domain is further described herein.

E3 Ligase Domain that Facilitates E2-Mediated Ubiquitination

An embodiment of present invention relates to a designer ubiquitin ligase or recombinant ubiquitin ligase that includes the antibody domain, as described herein and an E3 ligase domain. The E3 ligase domain facilitates ubiquitination of the complex. Ubiquitination occurs, in an embodiment, when an E2 enzyme interacts with a specific E3 partner and transfers the ubiquitin to the toxin active fragment. In some cases, it receives the ubiquitin from the E2 enzyme and transfers it to the target protein; in other cases, it acts by interacting with both the E2 enzyme and the toxin active fragment, but never itself receives the ubiquitin. With respect to the present invention, the antibody domain of the designer ubiquitin ligase binds to the toxin to form a complex between the polypeptide of the present invention and the toxin active fragment, and the E3 ligase domain allows for ubiquitination of the complex, which leads to the toxin's degradation.

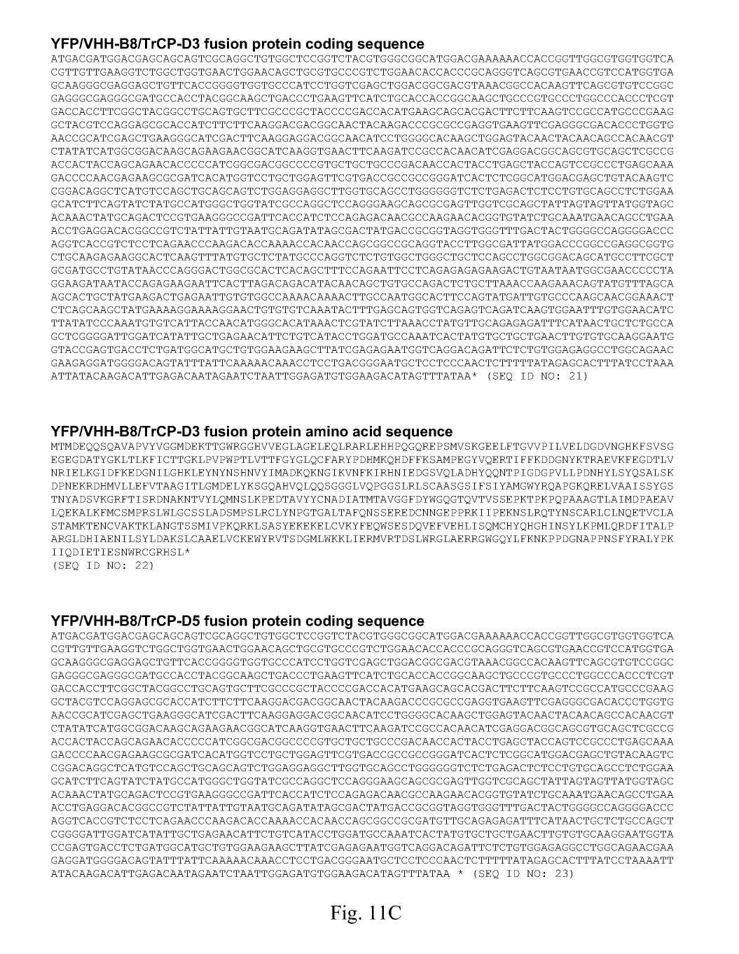

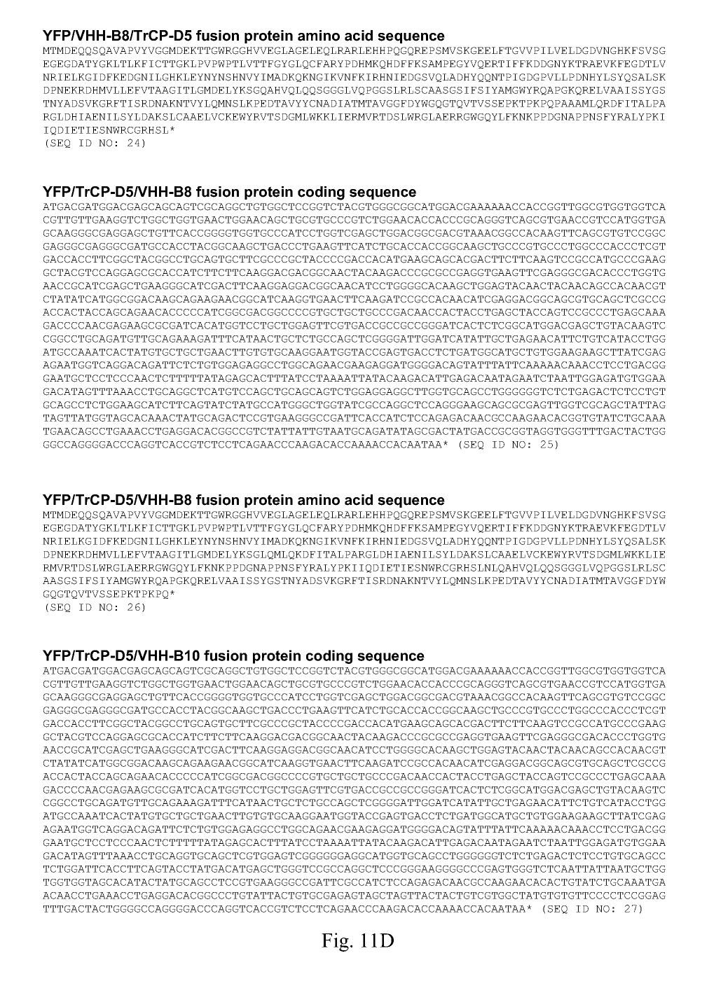

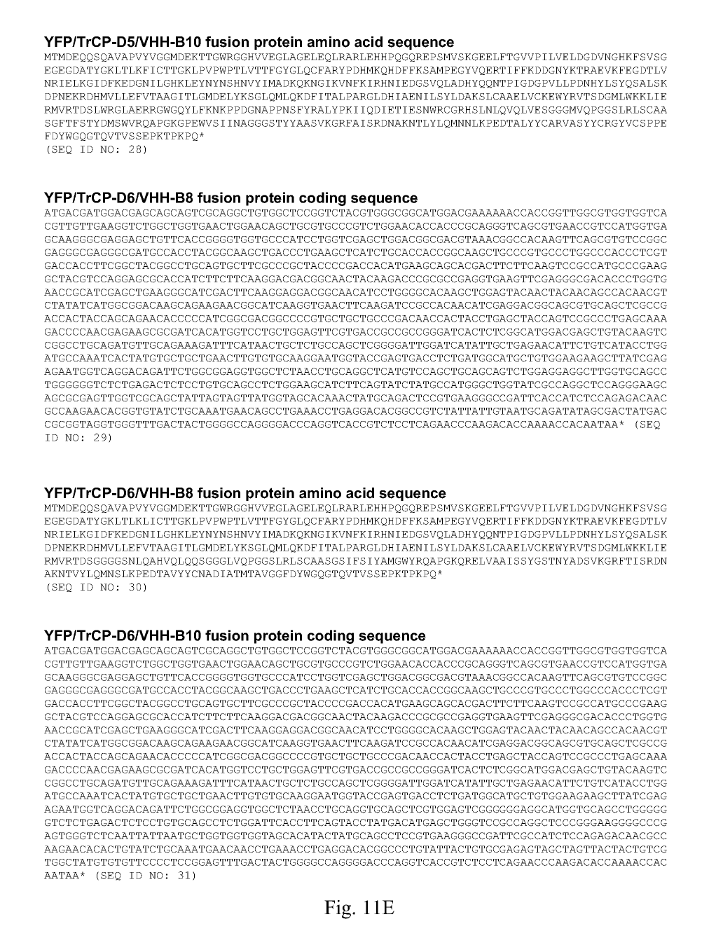

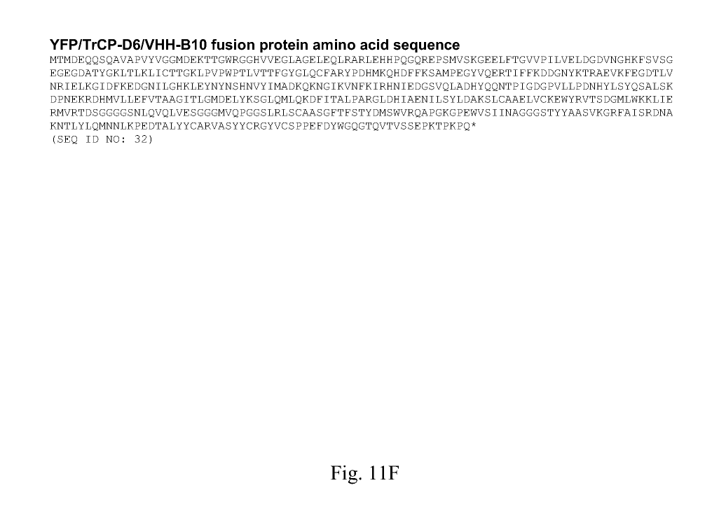

In particular, an E3 ligase can be a multimeric protein complex or a single chain protein that includes various complexes such as E2 binding domains. Examples of various E3 ligase domains include one or more RING, HECT, U-box, RIBRR, F-box domain, DCAF domain, HIFs-mimetic peptides, IkBs-mimetic sequences, BTB domain, or combination thereof. A domain referred to as TrCP was used as the E3 ligase domain in the present invention, and its nucleic acid sequence is shown in FIGS. 11A-11F as SEQ ID NO: 17. See Exemplification.

“Facilitating ubiquitination” refers to aiding or assisting in the attachment of one or more ubiquitin monomers via E2, which in turn, serves as a recognition site for proteasomal degradation. Without ubiquitination, the toxin will eventually degrade but takes a longer time, as compared to degradation via the assisted ubiquitination process. Accordingly, the presence of the E3 ligase, in an embodiment, allows for faster degradation of the toxin, as compared to degradation of the toxin via natural processes.

### Delivery Vehicles, Translocation and Cellular Binding Domains

In an embodiment of the present invention, the designer ubiquitin ligase has a domain which facilitates E2-mediated degradation of the toxin active fragment; and, in an embodiment, contains any or all of the following domains: a portion of the enzymatically active domain of a toxin rendered atoxic by one or more mutations, a translocation domain and a cell binding domain which binds to the cellular membrane of a cell and facilitates delivery the polypeptide molecule into the cytosol of the cell. The domains which are formed by any or all of a portion of the enzymatically active domain, the translocation domain and/or the cell binding domain are also referred to as a “delivery vehicle” and can be used interchangeably. The translocation and cellular binding (TCB) domains, in an embodiment, are fused or otherwise attached, directly or indirectly, to the antibody domain, as described herein and is referred to as cargo domains. The polypeptide of the present invention can further include the E3 ligase domain, also described herein as cargo.

In an embodiment, the TCB domains can be the heavy chain, chain “B” or the otherwise cell binding/translocation domain of a toxin. For example, the following portions or all of the following toxin proteins can be used as the TCB domain: BoNT serotype, Tcd A, Tcd B, atoxic Tcd. The heavy chain of a toxin can bind the cellular membrane of the intoxicated cell, and allow the transport of the molecule of the present invention as cargo into the inside of the cell.

In yet another embodiment, an atoxic holotoxin can be used to deliver the molecule of the present invention into the cell. A holotoxin is the entire toxin, both the light (e.g., the enzymatically active portion) and heavy (e.g., the TCB portion) chains of the toxin. See FIG. 9. An atoxic holotoxin refers to a molecule having both chain types, but mutated so that the light chain is no longer enzymatically active. Portions of the atoxic holotoxins can be mutated or deleted in any number of ways using methods known in the art. In the BoNT embodiment, serotype A is mutated/truncated to render the light chain enzymatically inactive. The nucleic acid (SEQ ID NO: 33) and amino acid (SEQ ID NO: 34) sequence of the light chain of BoNT/A is shown in FIG. 12A and examples of such deletions are shown in FIGS. 12B-C, as SEQ ID NO: 35-40. In a certain embodiment, portions of the toxin's light chain are deleted or truncated to render it inactive. In yet another embodiment, the heavy chain of the toxin can further be mutated, altered, and/or truncated. The amino acid sequence of the heavy chain of BoNT/A is shown in FIG. 12D as SEQ ID NO: 42. The first 4 amino acids of the sequence can be deleted, as an example. Methods of altering nucleic acid molecules are further described herein. The use of one type of delivery system that utilizes mutated light chain BoNT holotoxin is described in U.S. Pat. No. 6,203,794.

The composition of the present invention, the TCB domain can be, for example, covalently linked to the antibody domain and/or the E3 ligase domain and further can be, for example, a protein, peptide or peptidomimetic. In one embodiment, the composition of the present invention is a chimeric protein, peptide or peptidomimetic in which the delivery agent is operatively fused having e.g., a length of at most 50 or 100 residues.

A variety of TCB domains can be covalently linked to the antibody domain and/or E3 ligase domain and include, e.g., an antennapedia protein or active fragment thereof, such as an active fragment having the amino acid sequence RQIKIWFQNRRMKWKK (SEQ ID NO: 43); an HIV TAT protein or active fragment thereof, such as an active fragment having the amino acid sequence YGRKKRRQRRR (SEQ ID NO: 44); or a herpes simplex virus VP22 protein or active fragment thereof.

In general, in the composition of the present invention, TCB domain can be, for example, covalently linked to the domains described herein, as a protein, peptide or peptidomimetic. In one embodiment, the present invention is a chimeric protein, peptide or peptidomimetic in which the delivery agent is operatively fused to the antibody domain and/or E3 ligase domain. Such a composition can be, for example, a peptide or peptidomimetic having a length of at most 50 or 100 residues. Examples of penetratin-derived peptides that are useful as delivery agents include SEQ ID NO: 45 (RQIKIWFQNRRMKWKK), SEQ ID NO: 46 (KKWKMRRNQFWIKIQR); SEQ ID NO: 47 (RQIKIWFQNRRMKWKK); SEQ ID NO: 48 (RQIKIWFPNRRMKWKK); SEQ ID NO: 49 (RQPKIWFPNRRMPWKK); SEQ ID NO: 50 (RQIKIWFQNMRRKWKK); SEQ ID NO: 51 (RQIRIWFQNRRMRWRR); and SEQ ID NO: 52 (RRWRRWWRRWWRRWRR).

In another embodiment, the composition of the present invention includes a delivery agent which is a HIV trans-activator (TAT) protein or an active fragment thereof. Such a delivery agent can include, for example, a sequence identical or similar to residues 47-57 or 47-59 of HIV TAT. As an example, fusion proteins including residues 47-57 of HIV TAT (YGRKKRRQRRR; SEQ ID NO: 44) cross the plasma membrane of, for example, human and murine cells in vitro and in vivo; a variety of proteins from 15 to 120 KDa have been shown to retain biological activity when fused to a HIV TAT delivery agent. An HIV TAT delivery agent can be positively charged and can function, for example, in an energy-, receptor-, transporter- and endocytosis-independent manner to deliver a covalently linked antibody domain and/or E3 ligase domain to cells intoxicated with a toxin.

Yet another TCB domain for use with the present invention includes a herpes simplex virus VP22 protein or active fragment thereof. In a particular embodiment, the composition of the present invention includes an HSV type 1 (HSV-1) VP22 protein or active fragment thereof. HSV VP22, a nuclear transcription factor, can cross the plasma membrane through non-classical endocytosis and can enter cells independent of GAP junctions and physical contacts. As a fusion with a variety of different proteins, HSV VP22 results in uptake into cells of different types including terminally differentiated cells and can function to deliver a linked antibody domain and/or E3 ligase domain.

An example of another delivery agent useful in the present invention corresponds to or is derived from a hydrophobic signal sequence. Such a delivery agent can be, for example, the Kaposi fibroblast growth factor (kFGF) or an active fragment thereof such as AAVALLPAVLLALLAP (SEQ ID NO: 53); human β3 integrin or an active fragment thereof; or another hydrophobic delivery agent known in the art.

A delivery agent that can form a portion of the composition of the present invention also can be a synthetic sequence that shares one or more characteristics of a naturally occurring delivery agent such as a protein transduction domain (PTD). Such delivery agents include, but are not limited to, L- and D-arginine oligomers, for example, 9-mers of L- or D-arginine and related peptoids. Such delivery agents further include basic peptides and peptidomimetics; basic α-helical peptides and peptidomimetics; and peptides and peptidomimetics with optimized arginine alignment or optimized α-helical character as compared to a naturally occurring protein transduction domain such as residues 47-57 of HIV TAT. See, for example, WO 99/29721. Additional examples of delivery agents useful in the invention include SCWKₙ; (LARL)ₙ; HA2; RGD; K₁ 6RGD; oligomer; AlkCWK₁₈; DiCWK18; DipaLytic; Plae; Kplae and other delivery agents known in the art or developed in the future.

A delivery agent useful in the present invention also can be an agent that enables or enhances cellular uptake of the domains of the composition of the present invention that are associated non-covalently. In one embodiment, such a delivery agent is peptide containing two independent domains: a hydrophobic domain and a hydrophilic domain. In another embodiment, such a delivery agent is an MPG peptide, which is a peptide derived from both the nuclear localization sequence (NLS) of SV40 large T antigen and the fusion peptide domain of HIV-1 gP₄₁. In a further embodiment, such a delivery agent is an MPG peptide having the amino acid sequence GALFLGFLGAAGSTMGAWSQPKSKRKV (SEQ ID NO: 54). In yet a further embodiment, such a delivery agent is an amphipathic peptide such as Pep-1. These and related delivery agents that function in the absence of covalent linkage, also referred to as “protein transfection products,” can be used as the delivery system or the “TCB” domain of the present invention. Such peptide delivery agents/TCB domains for use with the composition of the present invention can be prepared by methods known in the art and/or are commercially available; as an example, the Chariot™ product is available from Active Motif (Carlsbad, Calif.).

### Methods of Inhibiting a Toxin

The present invention also relates to inhibiting one or more toxins, toxin active fragments, or toxin serotypes by contacting the composition of the present invention with the toxin. In particular, these methods are applicable in vitro and in vivo.

In vivo, the composition of the present invention is contacted with a cell intoxicated with the toxin active fragment that is the target of the antibody domain of the composition. The cell can be intoxicated using methods known in the art. For example, the cell can be intoxicated if the toxin is the holotoxin, or has the enzymatically active portion of the toxin fused with a cell binding/translocation domain. For example, the light chain of the BoNT/A can be delivered to a cell in any number of ways known in the art, and include exposing the enzymatically active portion of the toxin to the cell in concentrations and subjecting the mixture to conditions that allow entry of the toxin active fragment into the cell.

After the cell is intoxicated with the toxin active fragment or toxin, the intoxicated cell is exposed to or comes into contact with the composition of the present invention. The antibody domain binds the toxin active fragment and the E3 ubiquitin ligase domain facilitates polyubiquitination and the subsequent degradation of the toxin. The composition of the present invention is exposed to the intoxicated cell in an amount ranging from about 1 pM to 100 mM and for a length of time ranging from about 1 hr to 1 wk.

In vivo, the composition of the present invention is administered to an individual exposed to the toxin. The toxin enters cells of the individual and often causes paralysis or other symptoms depending on the type of toxin. The present invention includes methods of administering one or more designer ubiquitin ligases, described herein, to an individual. The antibody domain binds to the toxin active fragment that has intoxicated the cells of the individual. The amount of recombinant ubiquitin ligase of the present invention can be administered to the individual ranges from about 100 ng to about 5 gm.

In one aspect, the present invention embodies targeting multiple toxins or toxin serotypes. This can be accomplished at least in two ways using the compositions of the present invention. The composition of the present invention can include more than one antibody domains, e.g., a number of Camelid VHH domains, each specific to a different portion of the same toxin active fragment or to different toxin active fragments of different toxins. In another embodiment, multiple designer ubiquitin ligases, each to target different areas of one or more toxins are administered. In an embodiment, two, three or more different enzymatically active portions of a toxin or toxin serotypes (e.g., BoNT serotype A, B, C, etc.), can be used as the target for the antibody domain. In a case in which a number of serotypes can be involved in causing a disease or condition, such as botulism, multiple enzymatic portions of the toxin serotypes can be targeted. In the case of botulism, since any one of at least seven neurotoxin serotypes could be responsible for botulism, a molecule having with an antibody-binding domain to one, all, or any combination of the BoNT serotypes is encompassed by the present invention.

Alternatively, a pool of designer ligases can be prepared that contains an antibody domain that is specific for the enzymatic portion of one or more known serotypes that cause human disease. Botulism is often caused by exposure to a single BoNT serotype, but it is generally difficult to quickly determine which serotype is the cause. Thus, the standard of care in treating botulism includes administration of a number of antibodies to protect against most if not all of the serotypes that cause the disease in human. Hence, to protect against such a disease, an embodiment of the present invention includes having a cocktail of more than one designer ligase so that the ligases bind to several or preferably all of the serotypes that cause botulism. In such a case, the E3-ligase domain, as further described herein, can remain constant among the various designer ligases, whereas in another embodiment, they can differ.

The methods of the present invention include treating an individual infected with one or more toxins. This is accomplished by administering the designer ubiquitin ligase of the present invention to the infected individual. Administration ameliorates or reduces the severity of one or more the symptoms of the disease or condition. The presence, absence or severity of symptoms can be measured using tests and diagnostic procedures known in the art. Similarly the presence, absence and/or level of the toxin can be measured using methods known in the art. Symptoms or levels of the toxin can be measured at one or more time points (e.g., before, during and after treatment, or any combination thereof) during the course of treatment to determine if the treatment is effective. A decrease or no change in the level of the disease agent, or severity of symptoms associated therewith indicates that treatment is working, and an increase in the level of the toxin, or severity of symptoms indicates that treatment is not working Symptoms and levels of toxin are measured using methods known in the art.

In another embodiment, a formulation of the present invention can contain one or more of the DNA molecules that encode the designer ubiquitin ligase or portion thereof either present as a mixture or in the form of a DNA fusion molecule, each DNA molecule encoding a polypeptide as described above, such that the polypeptide is generated in situ. The DNA of the present invention can be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal).

In a preferred embodiment, the DNA can be introduced using a viral expression system (e.g., vaccinia or other pox virus, retrovirus, lentivirus, adenovirus, adeno-associated virus, alpha virus, baculovirus, or other viral vectors), which can involve the use of a non-pathogenic (defective), replication competent virus. In particular, adeno-associated viral delivery can be used to deliver nucleic acid molecules that encode the designer ubiquitin of the present invention. Techniques for incorporating DNA into such expression systems are well known to those of ordinary skill in the art. The DNA can also be “naked,” as described, for example, in Ulmer et al., Science 259:1745-1749, 1993 and reviewed by Cohen, Science 259:1691-1692, 1993. The uptake of naked DNA can be increased by coating the DNA onto biodegradable beads, which are efficiently transported into the cells.

The designer ubiquitin ligase of the present invention can be administered in one or more pharmaceutical carriers. The terms “pharmaceutically acceptable carrier” or a “carrier” refer to any generally acceptable excipient or drug delivery device that is relatively inert and non-toxic. The designer ubiquitin ligase of the present invention can be administered with or without a carrier. Exemplary carriers include calcium carbonate, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.), the teachings of which are incorporated herein by reference in their entirety. The ubiquitin designer ligase of the present invention can be administered systemically or locally (e.g., by injection or diffusion).

Suitable carriers (e.g., pharmaceutical carriers) also include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active substances, e.g., enzyme inhibitors, to reduce metabolic degradation. A carrier (e.g., a pharmaceutically acceptable carrier) is preferred, but not necessary to administer one or more designer ubiquitin ligases.

The designer ubiquitin ligase of the present invention can be administered intravenously, parenterally, orally, nasally, by inhalation, by implant, by injection, or by suppository. The composition can be administered in a single dose or in more than one dose over a period of time to confer the desired effect.

The actual effective amounts of compositions of the present invention can vary according to the designer ubiquitin ligase being utilized, the particular composition formulated, the mode of administration and the age, weight and condition of the patient, for example. As used herein, an effective amount of the designer ubiquitin ligase of the present invention is an amount which is capable of reducing one or more symptoms of the disease or conditions caused by the toxin. Dosages for a particular patient can be determined by one of ordinary skill in the art using conventional considerations, (e.g. by means of an appropriate, conventional pharmacological protocol).

The administration of the composition of the present invention and other anti-toxin drugs can occur simultaneously or sequentially in time to confer the desired effect.

Systems or kits of the present invention include one or more designer ubiquitin ligase of the present invention, as described herein.

### Polypeptides, Nucleic Acid Sequences, Vectors, Host Cells of Designer Ubiquitin Ligase of the Present Invention

As used herein, the term “recombinant” refers to a molecule that is one that is genetically made using techniques described herein. The present invention relates to a “recombinant” ubiquitin ligase or portions thereof that are engineered genetically.

As described in the Exemplification section, Camelid VHH antibodies specific to one of the botulinum neurotoxins, serotype A (BoNT/A) were made. Camelid VHH sequences engineered to bind to BoNT/A and BoNT/B are shown in FIGS. 10A-B. Specifically, the present invention relates to recombinant designer ubiquitin ligases having an antibody domain with the amino acid sequence of SEQ ID NO: 2, 4, 6, 8, 10, 12, 14, or 16 or combination thereof. Similarly, the present invention also includes a composition having an antibody domain that is encoded by a nucleic acid sequence of SEQ ID NO: 2, 4, 6, 8, 10, 12, 14, or 16 or combination thereof.

The present invention relates to isolated polypeptide molecules that have an antibody domain that has been engineered or isolated to act as inhibit the enzymatically active portion of the toxin. In particular, the present invention includes polypeptide molecules that contain the sequence of any one of the Camelid VHH antibodies specific to the light chain of BoNT/A and BoNT/B (SEQ ID NO: 2, 4, 6, 8, 10, 12, 14, 16, or combinations thereof). See FIGS. 10A-B. The present invention also pertains to polypeptide molecules that are encoded by nucleic acid sequences, SEQ ID NO: 1, 3, 5, 7, 9, 11, 13, 15, or combinations thereof).

As used herein, the term “polypeptide” encompasses amino acid chains of the designer ubiquitin ligase having any length, partial (e.g., antibody fragments) or full length proteins, wherein the amino acid residues are linked by covalent peptide bonds. Thus, a polypeptide can comprise a portion of the designer ubiquitin ligase or domain thereof, such as an antibody fragment, variable heavy chains, variable light chains and constant regains, or it can contain additional sequences. Note that terms, “heavy chain” and “light chain”, can be used describe a portion of the antibody fragment being used, but also can refer to the domain of the toxin (e.g., the light chain is the enzymatically active portion, and the heavy chain is the translocation/cell binding domain), depending on the context the term is being used. In a preferred embodiment, the antibody includes essentially the variable heavy chains that are specific to the enzymatically active portion of the toxin.

The polypeptides of the present invention referred to herein as “isolated” are polypeptides that are separated away from other proteins and cellular material of their source of origin. The compositions and methods of the present invention also encompass variants of polypeptides and DNA molecules of the present invention. A polypeptide “variant,” as used herein, is a polypeptide that differs from the recited polypeptide only in conservative substitutions and/or modifications, such that the ability of the designer ubiquitin ligase is retained.

The present invention also encompasses proteins and polypeptides, variants thereof, or those having amino acid sequences analogous to the amino acid sequences of binding agents described herein. Such polypeptides are defined herein as analogs (e.g., homologues), or mutants or derivatives. “Analogous” or “homologous” amino acid sequences refer to amino acid sequences with sufficient identity of any one of the amino acid sequences of the present invention so as to possess the biological activity (e.g., the ability to bind to the toxin). For example, an analog polypeptide can be produced with “silent” changes in the amino acid sequence wherein one, or more, amino acid residues differ from the amino acid residues of any one of the sequence, yet still possesses the function or biological activity of the polypeptide. In particular, the present invention relates to homologous polypeptide molecules having at least about 70% (e.g., 75%, 80%, 85%, 90% or 95%) identity or similarity with SEQ ID NO: 2, 4, 6, 8, 10, 12, 14, 16, 20, 22, 24, 26, 28, 30, 32, or combination thereof. Percent “identity” refers to the amount of identical nucleotides or amino acids between two nucleotides or amino acid sequences, respectfully. As used herein, “percent similarity” refers to the amount of similar or conservative amino acids between two amino acid sequences.

Homologous polypeptides can be determined using methods known to those of skill in the art. Initial homology searches can be performed at NCBI against the GenBank, EMBL and SwissProt databases using, for example, the BLAST network service. Altschuler, S. F., et al., J. Mol. Biol., 215:403 (1990), Altschuler, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 73:237-244 (1988) e.g., using default parameters).

The present invention, in one embodiment, includes an isolated nucleic acid molecule having a sequence of SEQ ID NO: 1, 3, 5, 7, 9, 11, 13, 15, 19, 21, 23, 25, 27, 29, 31, 35, 37, 39, 41, or combinations thereof. See FIGS. 10A-B and 11A-F. As used herein, the terms “DNA molecule” or “nucleic acid molecule” include both sense and anti-sense strands, cDNA, genomic DNA, recombinant DNA, RNA, and wholly or partially synthesized nucleic acid molecules. A nucleotide “variant” is a sequence that differs from the recited nucleotide sequence in having one or more nucleotide deletions, truncations, substitutions or additions. Such modifications can be readily introduced using standard mutagenesis techniques, such as oligonucleotide-directed site-specific mutagenesis as taught, for example, by Adelman et al. (DNA 2:183, 1983). Nucleotide variants can be naturally occurring allelic variants, or non-naturally occurring variants. Variant nucleotide sequences preferably exhibit at least about 70%, more preferably at least about 80% and most preferably at least about 90% homology to the recited sequence. Such variant nucleotide sequences will generally hybridize to the recited nucleotide sequence under stringent conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses isolated nucleic acid sequences that encode the antibody fragment and in particular, those which encode a polypeptide molecule having an amino acid sequence of SEQ ID NO: 2, 4, 6, 8, 10, 12, 14, 16, 20, 22, 24, 26, 28, 30, 32, 36, 38, 40, 42 or combinations thereof.

As used herein, an “isolated” nucleotide sequence is a sequence that is not flanked by nucleotide sequences which normally (e.g., in nature) flank the gene or nucleotide sequence (e.g., as in genomic sequences) and/or has been completely or partially purified from other transcribed sequences (e.g., as in a cDNA or RNA library). Thus, an isolated gene or nucleotide sequence can include a gene or nucleotide sequence which is synthesized chemically or by recombinant means. Nucleic acid constructs contained in a vector are included in the definition of “isolated” as used herein. Also, isolated nucleotide sequences include recombinant nucleic acid molecules and heterologous host cells, as well as partially or substantially or purified nucleic acid molecules in solution. The nucleic acid sequences that encode the antibody fragment of the present invention include homologous nucleic acid sequences. “Analogous” or “homologous” nucleic acid sequences refer to nucleic acid sequences with sufficient identity of any one of the nucleic acid sequences described herein, such that once encoded into polypeptides, they possess the biological activity of any one of the antibody fragments herein. In particular, the present invention is directed to nucleic acid molecules having at least about 70% (e.g., 75%, 80%, 85%, 90% or 95%) identity with SEQ ID NO: SEQ ID NO: 1, 3, 5, 7, 9, 11, 13, 15, 19, 21, 23, 25, 27, 29, 31, 35, 37, 39, 41, or combinations thereof.

Also encompassed by the present invention are nucleic acid sequences, DNA or RNA, which are substantially complementary to the DNA sequences encoding the polypeptides of the present invention, and which specifically hybridize with their DNA sequences under conditions of stringency known to those of skill in the art. As defined herein, substantially complementary means that the nucleic acid need not reflect the exact sequence of the sequences, but must be sufficiently similar in sequence to permit hybridization with nucleic acid sequence under high stringency conditions. For example, non-complementary bases can be interspersed in a nucleotide sequence, or the sequences can be longer or shorter than the nucleic acid sequence, provided that the sequence has a sufficient number of bases complementary to the sequence to allow hybridization therewith. Conditions for stringency are described in e.g., Ausubel, F. M., et al., Current Protocols in Molecular Biology, (Current Protocol, 1994), and Brown, et al., Nature, 366:575 (1993); and further defined in conjunction with certain assays.

### Stringency Conditions for Nucleic Acids:

Specific hybridization can be detected under high stringency conditions. “Stringency conditions” for hybridization is a term of art which refers to the conditions of temperature and buffer concentration which permit and maintain hybridization of a particular nucleic acid to a second nucleic acid; the first nucleic acid may be perfectly complementary to the second, or the first and second may share some degree of complementarity which is less than perfect. For example, certain high stringency conditions can be used which distinguish perfectly complementary nucleic acids from those of less complementarity. “High stringency conditions” for nucleic acid hybridizations and subsequent washes are explained, e.g., on pages 2.10.1-2.10.16 and pages 6.3.1-6 in Current Protocols in Molecular Biology (Ausubel, et al., In: Current Protocols in Molecular Biology, John Wiley & Sons, (1998)). The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in the art (Krause, M. H., et al., 1991, Methods Enzymol. 200:546-556). Also, low and moderate stringency conditions for washes are described (Ausubel, et al., In: Current Protocols in Molecular Biology, John Wiley & Sons, (1998)). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tm of about 17° C. Using these guidelines, the washing temperature can be determined empirically for high stringency, depending on the level of the mismatch sought. In some embodiments, high stringency conditions include those in which nucleic acid with less than a few mismatches does not bind. High stringency conditions, using these guidelines, lie in a temperature range between about 40° C. and about 60° C., an SSC concentration range between about 1× and about 10× (e.g., about 2×), and a reaction time range of between about 30 seconds and about 36 hours.

The present invention also provides vectors, plasmids or viruses containing one or more of the nucleic acid molecules having the sequence of SEQ ID NO: SEQ ID NO: 1, 3, 5, 7, 9, 11, 13, 15, 19, 21, 23, 25, 27, 29, 31, 35, 37, 39, 41 or combinations thereof). Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

### Peptidomimetic

Any polypeptide, or domain and/or portion thereof, described herein, can be substituted with a peptidomimetic. As used herein, the term “peptidomimetic” is used broadly to mean a peptide-like molecule that functions in the same manner as the polypeptides of the present invention. Such peptidomimetics include chemically modified peptides, peptide-like molecules containing non-naturally occurring amino acids, and peptoids, which are peptide-like molecules resulting from oligomeric assembly of N-substituted glycines, and function in a similar way as the toxin active fragment upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; an α,α-dialkyl-glycine or α-aminocycloalkane carboxylic acid; an N^{α}-C^{α} cyclized amino acid; an N^{α}-methylated amino acid; a β- or γ-amino cycloalkane carboxylic acid; an α,β-unsaturated amino acid; a β,β-dimethyl or β-methyl amino acid; a β-substituted-2,3-methano amino acid; an NC^{Δ} or C^{α}-C^{Δ} cyclized amino acid; or a substituted proline or another amino acid mimetic. In addition, a peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylenesulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

### Exemplification:

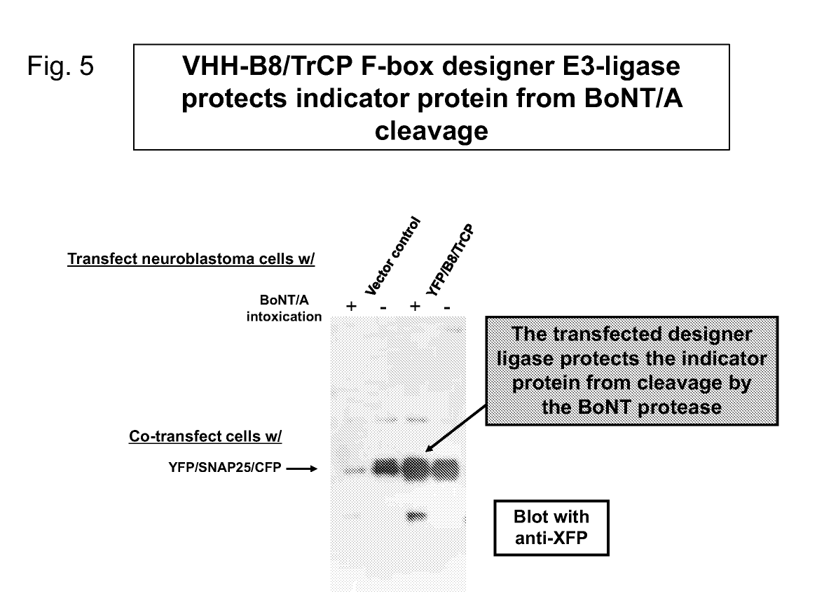

A vector was engineered for mammalian cell expression of a fusion protein containing yellow fluorescent protein (YFP) for detection, fused to the designer ligase in which VHH-B8 is joined to TrCP. The amino acid sequence of the resulting fusion protein (YFP/VHH-B8/TrCP) is shown in FIGS. 11A-F as SEQ ID NO: 20. This vector was co-transfected into neuronal cells (i.e., Neuro2a cells) using standard lipofection methods together with a second expression vector for a fusion protein consisting of YFP fused to SNAP25 and cerulean fluorescent protein (YFP/SNAP25/CFP). Following co-transfection, the cells were intoxicated with BoNT/A by normal methods and a day later the cells were lysed and the proteins resolved by SDS-PAGE and Western blotted with an antibody recognizing fluorescent proteins, specifically anti-XFP antibody recognizing the YFP component of these recombinant proteins. FIG. 5 shows that, while YFP/SNAP25/CFP is cleaved following BoNT/A intoxication in control cells, the presence of the VHH-B8/TrCP designer ligase led to much reduced levels of cleavage. This strongly indicates that the designer ligase led to the destruction of the intoxicating BoNT/A Lc protease. The result was apparently not due to inhibition of the protease by the VHH-B8 because other fusion proteins with VHH-B8 did not protect the YFP/SNAP25/CFP substrate from cleavage.

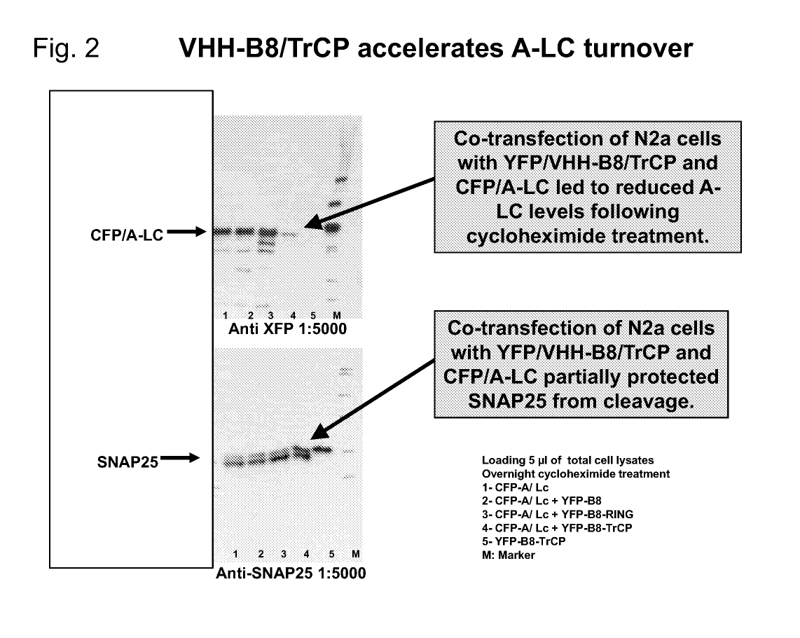

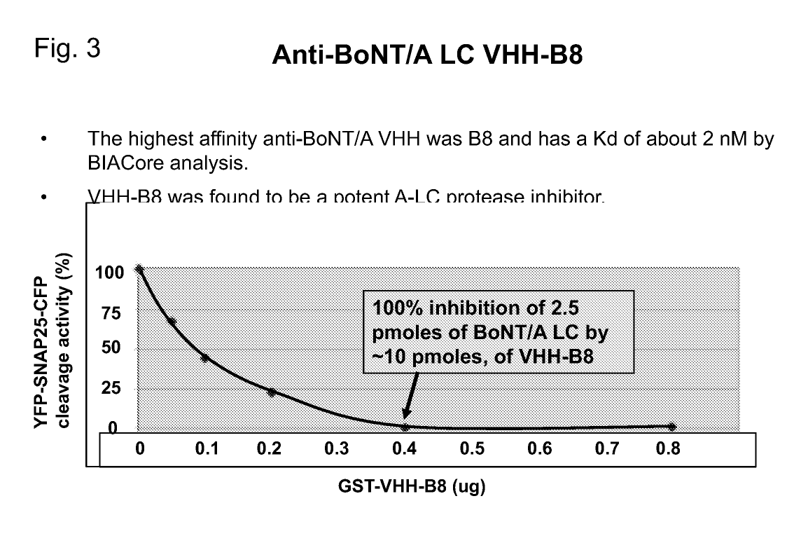

In another experiment, Neuro2a neuroblastoma cells were transfected with CFP fused to BoNT/A LC (CFP/A-LC). The cells were co-transfected with expression vector alone (YFP/VHH-B8) or expression vectors designed to express one of several fusion proteins (e.g., YFP/VHH-B8/RING or YFP/VHH-B8/TrCP). One of the fusion proteins was YFP fused only to the VHH-B8. Another fusion protein was YFP/VHH-B8/TrCP. A third fusion protein was YFP to the E3-ligase called RING. At the same time, cells were co-transfected with an expression plasmid for the CFP/A-LC protein. A day later, cells were treated with cycloheximide overnight and then extracts were prepared and resolved by SDS-PAGE for Western blot. The blot was probed with anti-XFP antibody recognizing the YFP component of these recombinant proteins. The results of FIG. 2 indicate that only the VHH-B8/TrCP fusion protein led to reduced levels of the co-transfected CFP/A-LC suggesting that degradation of A-LC is being accelerated by the designer ligase. In the cells co-transfected with VHH-B8/TrCP, the cellular substrate of BoNT/A protease, SNAP25, is less cleaved, also indicating that the VHH-B8/TrCP was protecting the substrate from cleavage by the protease, by accelerating its destruction.

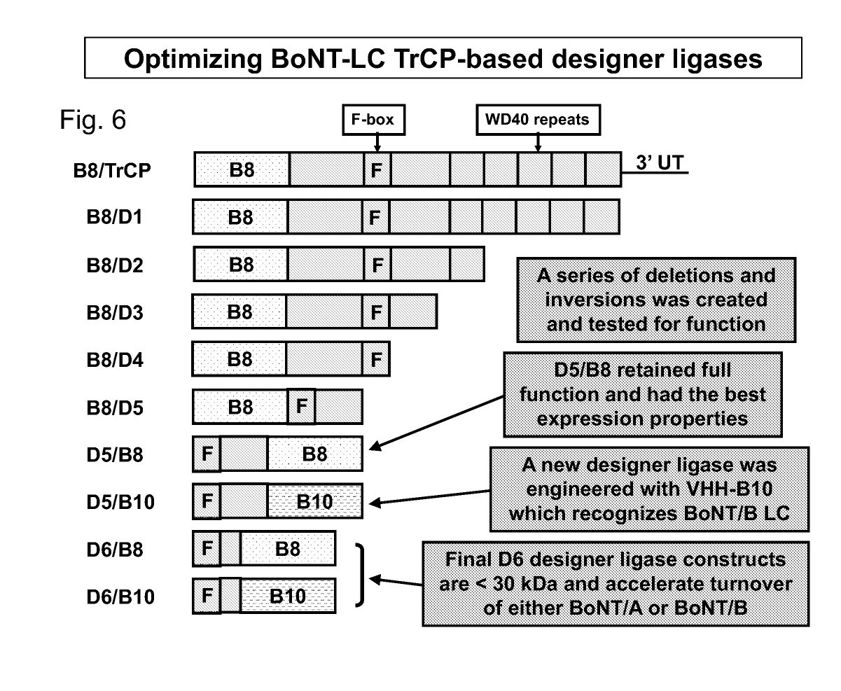

FIG. 6 provides a schematic representation of the series of deletions and inversions that were generated and tested for efficacy as designer ligases. B8 is the anti-BoNT/A Lc VHH-B8 and B10 is the anti-BoNT/B Lc VHH-B10. Because of the success of the VHH-B8/TrCP designer ligase, a series of four TrCP deletions (D1, D2, D3 and D4) containing the YFP and VHH-B8 domains fused to different regions of TrCP (B8/D1, B8/D2, B8/D3, B8/D4) were produced in which either the 3′ untranslated region of our previous B8/TrCP expression construction (D1), or this region plus variable amounts of the carboxyl end of TrCP were removed (D2, D3 and D4). The construction with the largest deletion (VHH-B8/TrCP-D4) lacks the entire TrCP carboxyl end beyond the F-box domain and is more than 40 kD smaller than the full-size B8/TrCP. Each construction was assayed by co-transfection of neuroblastoma cells with two mammalian expression vectors; one for a VHH-B8/TrCP (or control) and another to produce the YFP/SNAP25/CFP substrate protein. Following transfection, the cells were intoxicated by BoNT/A which results in the cleavage of the indicator protein. All four constructions had activity in protecting the SNAP25 indicator from BoNT/A cleavage. It appears that some of the activity was lost with the largest truncation, VHH-B8/TrCP-D4 compared to the somewhat smaller truncation, VHH-B8/TrCP-D3. The amino acid sequence of the YFP/VHH-B8/TrCP-D3 fusion protein is shown in FIGS. 11A-F, SEQ ID NO: 22. It was concluded that it is possible to remove most of the carboxyl end of TrCP without significantly reducing the ability of the protein to protect neuronal cells from BoNT/A intoxication measured by cleavage of the SNAP25 substrate.

Based on the initial results showing that the VHH-B8/D3 deletion was fully functional, a deletion of the amino coding end of TrCP was prepared in the VHH-B8/D3 expression vector. This expression construction was called B8/D5 and effectively produces a VHH-B8/TrCP fusion protein containing only the F-box domain of TrCP with. It is the F-box domain that is thought responsible for recruiting E3-ligase leading to ubiquitination and proteasome degradation of proteins bound to this protein. This construction was assayed, as above, for its function as a designer ligase to protect neuroblastoma cells from BoNT/A and it was found to as active as VHH-B8/D3 or the original VHH-B8/TrCP.

A complicated re-construction of the D3 and D5 deletions was performed in which the VHH-B8 was re-engineered at the carboxyl end of the TrCP coding DNA, an orientation more analogous to natural E3-ligases (see FIG. 6). These constructions were designated D3/VHH-B8 and D5/VHH-B8. Both proved to be at least as effective as the original B8/TrCP designer ligase. The amino acid sequences of the complete YFP/D3/VHH-B8 and YFP/D5/VHH-B8 are shown in FIGS. 11A-F, SEQ ID NO: 24 and SEQ ID NO: 26 respectively.

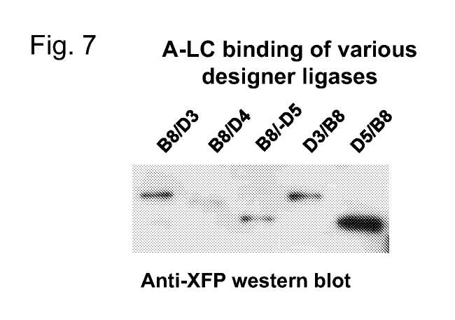

All of the different designer ligases were tested for the level of BoNT/A LC binding protein. Neuroblastoma cells were transfected with the different expression vectors and later cell extracts were prepared and incubated with GST/A-LC (A-LC fusion to glutathione-S-transferase). Proteins bound to A-LC were purified by affinity chromatography and a Western blot performed on the purified protein. The blot was probed with anti-XFP antibody recognizing the YFP component of these recombinant proteins. The results showed that the designer ligase constructions produced proteins of the appropriate size that bound to A-LC. The recovered designer ligase protein was readily detectable for D3, D4 and D5 TrCP deletions (FIG. 7). Note that VHH-B8/TrCP, VHH-B8/D1 and VHH-B8/D2 proteins could be observed with much longer exposure times at their appropriate molecular weights. The designer ligases with larger TrCP domains, (D1 and D2) were only detectable on long exposure and appeared to be heavily ubiquitinated, suggesting they are being rapidly degraded following synthesis in the cells. Most striking, the D5/VHH-B8 construction yielded substantially more A-LC binding protein than the others, including D3/VHH-B8 and B8/VHH-D5 (FIG. 7) suggesting that this was the most functional protein in terms of expression level and binding to A-LC.

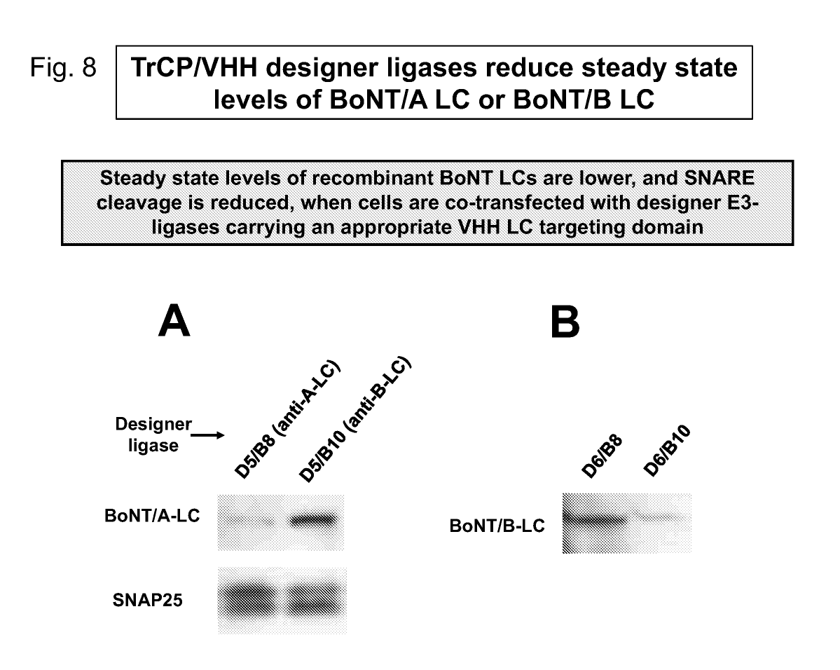

In light of the high functional expression level of the D5/VHH-B8 construction, and its apparent efficacy in protecting neuronal cell SNAP25 from cleavage following intoxication with BoNT/A, it was assessed whether the D5/VHH-B8 protein is a sufficiently efficient designer ligase to measurably reduce the steady-state levels of A-LC within neuronal cells transfected with an A-LC expression plasmid. In these studies, Neuro2a or M17 neuroblastoma cell lines were co-transfected with expression plasmids encoding a designer ligase and CFP/A-LC. A BoNT/B designer ligase was constructed in which the anti-BoNT/A-LC VHH-B8 component of D5/VHH-B8 was replaced by the anti-B-LC VHH-B10 (called D5/VHH-B10—See FIG. 6). The amino acid sequences of the complete YFP/D5/VHH-B10 is shown in FIGS. 11A-F, SEQ ID NO: 28. A day later, extracts were prepared and resolved by SDS-PAGE for Western blot. The blots were probed with anti-A-LC, anti-B-LC mAbs or anti-SNAP25 polyclonal antibodies. As shown in FIG. 8, the steady state level of A-LC was significantly lower in cells transfected with the expression plasmid for D5/VHH-B8 than in cells transfected with the expression plasmid for D5/VHH-B10 ligase. This analysis has been repeated at least five times, several times in triplicate, reproducibly demonstrating reduced steady-state levels of A1-LC in the cells co-transfected with D5/VHH-B8. Furthermore, when the cell extracts were blotted for SNAP25, a reduced level of cleavage in cells co-transfected with D5/VHH-B8 could be observed, as compared to those co-transfected with D5/VHH-B10 (FIG. 8A). In other studies not shown, co-transfected cells grown in the presence of the proteasome inhibitor, MG132, did not have reduced steady-state levels of A-LC, showing that the D5/VHH-B8 was reducing A-LC levels by a proteasome degradation-mediated process.

Because of the excellent results showing that the D5/B8 designer ligase caused reduced steady-state levels of A-LC, indicating accelerated proteasome-mediated turnover, an even further truncated version of TrCP, called D6, was designed and produced. It contains almost no TrCP coding DNA outside of the F-box domain (see FIG. 6). The D6 coding DNA was joined in frame with either the anti-A-LC VHH, B8, or the anti-B-LC VHH, B10. The resulting fusion proteins were called D6/VHH-B8 and D6/VHH-B10 respectively. As a possible further improvement, the D6 and the VHH domains were separated by DNA encoding a short flexible spacer protein, GGGGS, in an effort to improve domain functional independence and folding. The D6-based designer ligases were then tested as designer ligases in recent work. The amino acid sequences of the complete YFP/D6/VHH-B8 and YFP/D6/VHH-B10 is shown in FIGS. 11A-F, SEQ ID NO: 30 and SEQ ID NO: 32 respectively. The D6-based ligases have also proved effective in reducing the steady state levels of the BoNT LC targeted by the VHH. FIG. 8B shows the reduced steady levels of B-LC in cells cotransfected with CFP/B-LC and D6/B10 compared to D6/B8 (which targets A-LC). Thus by replacing the A-LC binding VHH-B8 with the B-LC binding VHH-B10 the new designer ligase accelerates turnover of BoNT/B protease in neuronal cells. This result shows the broad applicability of our approach to other BoNT serotypes and other toxins.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A recombinant ubiquitin ligase molecule that comprises:
a. an antibody fragment that is specific for a toxin active fragment, wherein the toxin active fragment is an enzymatically active fragment of one or more toxins or toxin serotypes; and
b. an E3-ligase domain that comprises an E3-ligase or polypeptide that facilitates E2-mediated ubiquitination of the toxin active fragment.

2. The recombinant ubiquitin ligase molecule of claim 1, wherein the toxin comprises botulinum neurotoxin (BoNT), Tcd A, Tcd B, Clostridium Lethal Toxin, Anthrax Lethal Factor, Ricin, Exotoxin A, Diphtheria, Cholera, Tetanus toxins, Shiga toxin, and a combination thereof.

3. The recombinant ubiquitin ligase molecule of claim 2, wherein the antibody is specific for a light chain protease domain of one or more botulinum neurotoxin (BoNT) serotypes (A-G) or tetanus toxin.

4. The recombinant ubiquitin ligase molecule of claim 2, wherein antibody is a Camelid antibody fragment, shark antibody fragment, or a lamprey antibody fragment.

5. The recombinant ubiquitin ligase molecule of claim 2, wherein antibody fragment is a single chain antibody fragment.

6. The recombinant ubiquitin ligase molecule of claim 2, wherein a polypeptide that facilitates E2-mediated ubiquitination comprises an antibody specific to E2.

7. The recombinant ubiquitin ligase molecule of claim 1, wherein the E3 ligase domain comprises one or more RING, HECT, IkB, HIF, U-box, RIBRR, F-box, BTB, or DDS2.

8. An isolated polypeptide molecule that comprises:
a. a Camelid VHH antibody fragment that is specific for a toxin active fragment, wherein the toxin active fragment is an enzymatically active fragment of one or more BoNT serotypes;
b. an enzymatically active domain of a toxin having one or more mutations, wherein a portion thereof is rendered atoxic by said one or more mutations;
c. a translocation domain; and
d. a cell binding domain binds to a cell and facilitates delivery of the polypeptide molecule into the cytosol of the cell.

9. The isolated polypeptide of claim 8, wherein the atoxic enzymatic domain, the translocation domain and/or the cell binding domain are comprised from a BoNT serotype, Tcd A, Tcd B, or shiga toxin.

10. The isolated polypeptide of claim 9, further comprising an E3-ligase domain that comprises an E3-ligase or polypeptide that facilitates E2-mediated degradation of the toxin active fragment.

11. A recombinant ubiquitin ligase molecule that comprises:
a. an antibody fragment that is specific for a toxin active fragment, wherein the toxin active fragment is an enzymatically active fragment of one or more botulinum neurotoxin (BoNT) serotypes; and
b. an E3-ligase domain that comprises an E3-ligase or polypeptide that facilitates E2-mediated degradation of the toxin active fragment.

12. A recombinant ubiquitin ligase molecule that comprises:
a. an antibody fragment that is specific for a toxin active fragment, wherein the toxin active fragment is an enzymatically active fragment of one or more botulinum neurotoxin (BoNT) serotypes;
b. an E3-ligase domain that comprises an E3-ligase or polypeptide that facilitates E2-mediated degradation of the toxin active fragment;
c. an enzymatically active domain of a toxin having one or more mutations, wherein a portion thereof is rendered atoxic by said one or more mutations and the portion includes 4 or more contiguous amino acids;
d. a translocation domain; and
e. a cell binding domain binds to the cellular membrane of a cell and facilitates delivers the polypeptide molecule into the cytosol of the cell.

13. The recombinant ubiquitin ligase molecule of claim 12, wherein the translocation/cellular binding domain comprises an antennapedia protein, HIV TAT protein, herpes simplex virus VP22 protein, penetratin-derived peptides, kFGF, human β3 integrin, L- and D-arginine oligomers, SCWKₙ, (LARL)ₙ, HA2; RGD; K₁ 6RGD oligomer; AlkCWK₁₈, DiCWK18, DipaLytic; Plae, Kplae, MPG peptide, Pep-1, or an atoxic neurotoxin.

14. A Camelid VHH antibody that is specific to the BoNT/A or BoNT/B light chain.

15. An isolated antibody that is specific to the BoNT/A or BoNT/B light chain, wherein the antibody has a sequence that comprises an amino acid sequence selected from the group consisting of:
a. an amino acid sequence encoded by a nucleic acid molecule having a sequence of SEQ ID NO: 1, 3, 5, 7, 9, 11, 13, or 15;
b. an amino acid sequence encoded by a complement of SEQ ID NO: 1, 3, 5, 7, 9, 11, 13, or 15;
c. an amino acid sequence encoded by a nucleic acid molecule that hybridizes to SEQ ID NO: 1, 3, 5, 7, 9, 11, 13, or 15; under high stringency conditions; and
d. an amino acid sequence of SEQ ID NO: 2, 4, 6, 8, 10, 12, 14, or 16.

16. An isolated antibody that is specific to the BoNT/A or BoNT/B light chain, wherein the antibody has a sequence that comprises an amino acid sequence selected from the group consisting of:
a. an amino acid sequence encoded by a nucleic acid having greater than or equal to about 70% identity with SEQ ID NO: 1, 3, 5, 7, 9, 11, 13, or 15;
b. an amino acid sequence encoded by a complement having greater than or equal to about 70% identity with of SEQ ID NO: 1, 3, 5, 7, 9, 11, 13, or 15;
c. an amino acid sequence encoded by a nucleic acid molecule having greater than or equal to about 70% identity with a molecule that hybridizes to SEQ ID NO: 1, 3, 5, 7, 9, 11, 13, or 15; and
d. an amino acid sequence having greater than or equal to about 70% similarity to a sequence set forth in SEQ ID NO: 2, 4, 6, 8, 10, 12, 14 or 16.

17. An isolated nucleic acid molecule that encodes an isolated antibody that is specific to the BoNT/A or BoNT/B light chain, wherein said antibody is encoded by a nucleic acid molecule having a sequence selected from the group consisting of:
a. SEQ ID NO: 1, 3, 5, 7, 9, 11, 13, or 15;
b. a complement of SEQ ID NO: 1, 3, 5, 7, 9, 11, 13, or 15;
c. that hybridizes to SEQ ID NO: 1, 3, 5, 7, 9, 11, 13, or 15 under high stringency conditions; and
d. that encodes SEQ ID NO: 2, 4, 6, 8, 10, 12, 14, or 16.

18. An isolated nucleic acid molecule that encodes an isolated antibody that is specific to the BoNT/A or BoNT/B light chain, wherein said antibody is encoded by a nucleic acid molecule having greater than or equal to about 70% identity with a sequence selected from the group consisting of:
a. SEQ ID NO: 1, 3, 5, 7, 9, 11, 13, or 15;
b. a complement of SEQ ID NO: 1, 3, 5, 7, 9, 11, 13, or 15;
c. that hybridizes to SEQ ID NO: 1, 3, 5, 7, 9, 11, 13, or 15 under high stringency conditions; and
d. that encodes SEQ ID NO: 2, 4, 6, 8, 10, 12, 14, or 16.

19. A host cell transfected with the nucleic acid sequence of claim 17.

20. A host cell transfected with a nucleic acid sequence that encodes the polypeptide of claim 15.

21. A vector or plasmid that comprises the nucleic acid molecule of claim 17.

22. An isolated polypeptide, wherein the polypeptide comprises:
a. a Camelid VHH antibody domain that is specific to the BoNT/A or BoNT/B light chain, wherein the Camelid VHH antibody domain has a sequence that comprises an amino acid sequence selected from the group consisting of:
i. an amino acid sequence encoded by a nucleic acid molecule having a sequence of SEQ ID NO: 1, 3, 5, 7, 9, 11, 13, or 15;
ii. an amino acid sequence encoded by a complement of SEQ ID NO: 11, 3, 5, 7, 9, 11, 13, or 15;
iii. an amino acid sequence encoded by a nucleic acid molecule that hybridizes to SEQ ID NO: 1, 3, 5, 7, 9, 11, 13, or 15 under high stringency conditions; and
iv. an amino acid sequence of SEQ ID NO: 2, 4, 6, 8, 10, 12, 14, or 16; and
b. a BoNT domain that comprises an amino acid sequence selected from the group consisting of:
i. an amino acid sequence encoded by a nucleic acid molecule having a sequence of SEQ ID NO: 33, 35, 37, 39, or 41;
ii. an amino acid sequence encoded by a complement of SEQ ID NO: 33, 35, 37, 39, or 41;
iii. an amino acid sequence encoded by a nucleic acid molecule that hybridizes to SEQ ID NO: 33, 35, 37, 39, or 41; and
iv. an amino acid sequence of SEQ ID NO: 34, 36, 38, 40, or 42.

23. An isolated polypeptide, wherein the polypeptide has a sequence that comprises an amino acid sequence selected from the group consisting of:
a. an amino acid sequence encoded by a nucleic acid having greater than or equal to about 70% identity with SEQ ID NO: 19, 21, 23, 25, 27, 29, 31, 35, 37, 39, or 41;
b. an amino acid sequence encoded by a complement having greater than or equal to about 70% identity with of SEQ ID NO: 19, 21, 23, 25, 27, 29, 31, 35, 37, 39, or 41;
c. an amino acid sequence encoded by a nucleic acid molecule having greater than or equal to about 70% identity with a molecule that hybridizes to SEQ ID NO: 19, 21, 23, 25, 27, 29, 31, 35, 37, 39, or 41; and
d. an amino acid sequence having greater than or equal to about 70% similarity to a sequence set forth in SEQ ID NO: 20, 22, 24, 26, 28, 30, 32, 36, 38, 40 or 42.

24. An isolated nucleic acid molecule, wherein the nucleic acid molecule has a sequence that comprises a nucleic acid sequence selected from the group consisting of:
a. a nucleic acid sequence having greater than or equal to about 70% identity with SEQ ID NO: 19, 21, 23, 25, 27, 29, 31, 35, 37, 39, or 41;
b. a nucleic acid sequence complement having greater than or equal to about 70% identity with of SEQ ID NO: 19, 21, 23, 25, 27, 29, 31, 35, 37, 39, or 41;
c. a nucleic acid molecule having greater than or equal to about 70% identity with a molecule that hybridizes to SEQ ID NO: 19, 21, 23, 25, 27, 29, 31, 35, 37, 39, or 41; and
d. a nucleic acid sequence that encodes an amino acid sequence having greater than or equal to about 70% similarity to a sequence set forth in SEQ ID NO: 20, 22, 24, 26, 28, 30, 32, 36, 38, 40 or 42.

25. A method of degrading or inhibiting one or more BoNT serotypes that have intoxicated one or more cells, the method comprises:
contacting an amount of the recombinant ubiquitin ligase of claim 1 with the intoxicated cells;
wherein the recombinant ubiquitin ligase inhibits and/or leads to the degradation of at least one BoNT serotype.

26. The method of claim 25, wherein an amount of recombinant ubiquitin ligase ranges from about 1 pM to about 100 mM.

27. The method of claim 26, wherein the recombinant ubiquitin ligase is contacted for a time that ranges from about 1 hour and about 1 week.

28. A method of treating an individual having one or more cells intoxicated with one or more BoNT serotypes, the method comprises:
administering to the individual an amount of recombinant ubiquitin ligase of claim 1 in a carrier;
wherein one or more symptoms associated with BoNT intoxication are reduced or reversed.

29. The method of claim 26, wherein one or more symptoms associated with BoNT intoxication that are reduced or revised include blurred vision, dry mouth, difficulty swallowing, difficulty speaking, paralysis, muscle weakness; respiratory failure, and decreased nerve conduction.

30. The method of claim 28, wherein the amount of recombinant ubiquitin ligase is administered intravenously, parenterally, orally, nasally, by inhalation, by implant, by injection, or by suppository.

31. The method of claim 30, wherein the amount of recombinant ubiquitin ligase is administered once or periodically.

32. A pharmaceutical composition comprising the recombinant ubiquitin ligase molecule of claim 1, and a carrier.
